(19) United States
(12) Patent Application Publication
(10) Pub. No.: US 2026/0253701 A1
(43) Pub. Date: Aug. 27, 2026

(54) EEG BIOMARKERS FOR ALPHA5 PAM DOSAGE DETERMINATION AND THERAPEUTIC MONITORING

(71) Applicant: Centre for Addiction and Mental Health, Toronto (CA)

(72) Inventors: Etay Hay, Toronto (CA); Etienne Sibille, Toronto (CA); Thomas D. Prevot, Toronto (CA)

(73) Assignee: Centre for Addiction and Mental Health, Toronto, ON (CA)

(21) Appl. No.: 19/127,470

(22) PCT Filed: Nov. 6, 2023
(86) PCT No.: PCT/IB2023/061190

§ 371 Date: May 6, 2025

Related U.S. Application Data
(60) Provisional application No. 63/382,577, filed on Nov. 7, 2022.

(51) Int. Cl. G16H 20/10; A61B 5/00; A61B 5/16; A61B 5/374; A61K 31/5517; A61P 25/24

(52) U.S. Cl. CPC G16H 20/10; A61B 5/165; A61B 5/374; A61B 5/4839; A61B 5/4848; A61K 31/5517; A61P 25/24

(57) ABSTRACT

The invention provides methods of dosing and monitoring efficacy of GABA_{A} receptor positive allosteric modulators (PAM) therapy, including α5-PAM therapy, using a patient's power spectral density (PSD) features via non-invasive electroencephalography.

This application claims priority to U.S. Provisional Application No. 63/382,577 filed Nov. 7, 2022, the contents of which are hereby incorporated by reference in their entirety.

### BACKGROUND

Recent studies implicate cellular and circuit mechanisms in depression, and reduced inhibition by somatostatin-expressing (SST) interneurons is a key component associated with treatment-resistant depression. Administration of positive allosteric modulators of GABA_{A} receptor alpha5 subunits (α5-PAM) that selectively target and recover this lost inhibition, exhibit antidepressant, anxiolytic, and pro-cognitive effects in rodents. However, the functional effects of this drug on human cortical activity in vivo are unknown, and currently cannot be readily assessed. Non-invasive biomarkers for assessing drug efficacy are needed. The present invention addresses this need.

### BRIEF SUMMARY

The disclosure provides electroencephalography (EEG) biomarkers for therapy with GABAA-positive allosteric modulators (PAM). In an aspect, the PAM is an α5-PAM. In aspects, the disclosure provides methods of determining a therapeutic dose of a PAM, such as an α5-PAM, for a subject in need thereof, the methods including obtaining or receiving an EEG signal from the subject, performing, on a processor, a power spectral density (PSD) calculation on the signal to obtain a raw PSD, decomposing, on a processor, the raw PSD into its aperiodic and periodic components, determining, based on the raw PSD, the aperiodic and periodic components, and a dose prediction function, the therapeutic dose of an α5-PAM for the subject.

The disclosure also provides methods of monitoring therapeutic efficacy of therapy of a GABAA-positive allosteric modulator (PAM) in a subject in need thereof, including an α5-PAM, the methods including obtaining or receiving an electroencephalography (EEG) signal from the subject in a pre-treatment condition and at least one subsequent post-treatment condition, where the pre-treatment condition corresponds to pre-treatment with the PAM, such as an α5-PAM, and the post-treatment condition corresponds to after administration of at least one dose of the PAM, such as an α5-PAM, to the subject, performing, on a processor, a power spectral density (PSD) calculation on the signal obtained or received for each condition to obtain raw PSD for each condition, decomposing, on a processor, the raw PSD into its aperiodic and periodic components for each condition, determining, based on the raw PSD, the aperiodic and periodic components, and a dose prediction function, a predicted dose for the subject for each condition; comparing the predicted dose at each condition, wherein, a decrease in predicted dose indicates that the α5-PAM is having a therapeutic effect; and, optionally, administering a second or further dose of the α5-PAM to the subject and repeating until the predicted dose is zero, or smaller than a predetermined amount.

In aspects, the methods may also include where the aperiodic components comprise broadband AUC and exponent (χ).

In aspects, the methods may also include where the raw PSD and periodic components comprise the power measured by integral of one or more PSD frequency bands selected from theta (4-8 Hz), alpha (8-12 Hz), and beta (12-30 Hz) frequency bands, and/or sub-bands thereof.

In aspects, the dose prediction function is:

$$Optimal Dose = 0.04 \cdot 1 / f + 0.08 \cdot θ + 0.13 \cdot α + 0.11$$

where input metrics are z-scored aperiodic components (1/f) of the PSD and power spectral density in the theta (θ) and alpha (α) frequency ranges, relative to healthy, and optimal dose is expressed as fraction relative to the reference dose.

In aspects, the predicted dose is for a pre-treatment condition and a post-treatment condition of the subject.

In aspects of any of the foregoing methods, the EEG signal is obtained over a period of time. The period of time may be from 2-5 minutes, from 5-15 minutes, from 15-30 minutes, from 30-60 minutes, or from 60-120 minutes. In aspects, the period of time may be about 5, 10, 15, 20, 25, 30, 35, 40, 45, 50, 60, 90, or 120 minutes.

In aspects of any of the foregoing methods, the α5-PAM is GL-II-73 or a derivative thereof.

In aspects of any of the foregoing methods, the subject in need includes one diagnosed with a neuropsychiatric disorder, a neurological disorder, a neurodegenerative disorder, or any combination thereof. In aspects, the neuropsychiatric disorder is bipolar disorder or schizophrenia. In aspects, the neurological disorder is epilepsy or other brain related disorders. In aspects, the neurodegenerative disorder is Alzheimer's disease or dementia. In aspects, the subject is human.

In aspects of any of the foregoing methods, the subject in need includes one who has been diagnosed with depression. In aspects, the subject has been diagnosed with major depressive disorder (MDD). In aspects, the depression or MDD is treatment resistant. In aspects, the subject is human.

### BRIEF DESCRIPTION OF THE DRAWINGS

FIG. 1A is a schematic illustration of the detailed human L2/3 pyramidal neuron model and the different experimental conditions (GABA—application of GABA, a5-PAM—application of the GL-II-73 compound, PTX—GABA block).

FIG. 1B is a bar graph showing simulated tonic inhibition current recordings at soma in the different conditions, fitted to reproduce the experimentally recorded current magnitude averages (experimental mean±standard deviation shown as error bars).

FIG. 1C is a bar graph showing derived apical tonic inhibition conductance values in the GABA (left bar) and GABA+α5-PAM (right bar) conditions, indicating a 60% modulation of apical tonic inhibition conductance by α5-PAM.

FIG. 1D is a schematic illustration of the model L2/3 circuit connectivity and summary of MDD and α5-PAM circuit effects.

FIG. 1E is a schematic illustration of the detailed L2/3 microcircuit models with human model morphologies (from top to bottom: Pyr, VIP, PV, SST).

FIG. 2A is an example raster plot of simulated baseline and response spiking in a healthy microcircuit model. Dashed line indicates stimulus time.

FIG. 2B is a bar graph showing baseline and response Pyr neuron firing rates in the different simulated conditions. α5-PAM restores healthy levels of baseline firing.

FIG. 2C is a bar graph showing signal to noise ratio (SNR) of response in each condition, where α5-PAM boosts SNR to healthy levels.

FIG. 2D illustrates the distributions of baseline and response firing rates (n=2,951 windows×200 microcircuits pre-stimulus, n=200 windows post-stimulus). The vertical lines denote the decision boundaries, and the shaded areas show the failed/false detections.

FIG. 2E is a bar graph showing probability of failed detection and false detection in each simulated condition. In each set of three bars, from left, conditions are Healthy, MDD, and MDD+α5-PAM. α5-PAM significantly reduced both failed and false detection rates, bringing them close to the healthy levels. All asterisks denote significant paired t-tests (p<0.05) with effect sizes greater than 1, when compared to healthy (black asterisks) or MDD (grey asterisks). N=200 randomized microcircuits per condition.

FIG. 3A is a bar graph showing Pyr neuron baseline and response firing rates for MDD (left-most bar in each set) and effect of different doses of α5-PAM corresponding to 25%, 50%, 75%, 100%, 125%, and 150% of the estimated therapeutic dose (left to right). The black horizontal lines and shaded areas denote the healthy mean±standard deviation. All asterisks denote significant paired t-tests (p<0.05) with effect sizes greater than 1, when compared to healthy (black asterisks) or MDD (grey asterisks). N=200 randomized microcircuits per condition.

FIG. 3B is a bar graph showing signal to noise ratio (SNR) for different doses of α5-PAM as in FIG. 3A.

FIG. 3C is a bar graph showing failed and false detection for the conditions in FIG. 3A.

FIG. 4A illustrates EEG signals generated from the human cortical microcircuit models.

FIG. 4B is a line graph showing bootstrapped mean, and 95% confidence intervals of power spectral densities (PSDs) of simulated EEG from microcircuit models from each condition as in FIG. 4A Top line is MDD, Bottom line is Healthy, Middle line is MDD+α5-PAM. α5-PAM restores PSD profile to healthy level.

FIG. 4C is a scatter plot showing power spectral density in the theta sub-band (4-8 Hz) for Healthy, MDD, and the indicated α5-PAM doses. Asterisks denote significant paired t-tests (p<0.05) with effect sizes greater than 1, when compared to healthy (black asterisks) or MDD (grey asterisks). N=50 randomized microcircuits per condition.

FIG. 4D is a scatter plot showing power spectral density in the alpha sub-band (8-12 Hz) for Healthy, MDD, and the indicated α5-PAM doses. Asterisks denote significant paired t-tests (p<0.05) with effect sizes greater than 1, when compared to healthy (black asterisks) or MDD (grey asterisks). N=50 randomized microcircuits per condition.

FIG. 4E is a scatter plot showing power spectral density in the beta sub-band (12-21 Hz) for Healthy, MDD, and the indicated α5-PAM doses. Asterisks denote significant paired t-tests (p<0.05) with effect sizes greater than 1, when compared to healthy (black asterisks) or MDD (grey asterisks). N=50 randomized microcircuits per condition.

FIG. 5A is a line graph showing aperiodic components of the PSD for each condition (healthy 504, MDD 502, and MDD+100% α5-PAM 506).

FIG. 5B is a scatter plot showing broadband power spectral density area under the curve (3-30 Hz; AUC or integral) of the aperiodic component of the PSD for healthy, MDD, and each α5-PAM dose.

FIG. 5C is a scatter plot showing exponent (χ) of the aperiodic component of the PSD for healthy, MDD, and each α5-PAM dose.

FIG. 5D is a line graph showing the periodic component of the PSD for each condition (healthy 504; MDD 502; MDD+100% α5-PAM 506).

FIG. 5E is a scatter plot showing power of the periodic component of PSD in theta band (θ, 4-8 Hz) for each α5-PAM dose. All asterisks denote significant paired t-tests (p<0.05) with effect sizes greater than 1, when compared to healthy (black asterisks) or MDD (grey asterisks). N=50 randomized microcircuits per condition.

FIG. 5F is a scatter plot showing power of the periodic component of PSD in beta band (B, 12-21 Hz) for each α5-PAM dose. All asterisks denote significant paired t-tests (p<0.05) with effect sizes greater than 1, when compared to healthy (black asterisks) or MDD (grey asterisks). N=50 randomized microcircuits per condition.

FIG. 6A is a schematic illustration of non-selective PAM effects on the model connectivity.

FIG. 6B is a bar graph showing baseline and response Pyr neuron firing rates in the different conditions, from left in each set of three bars: healthy, MDD, MDD+benzodiazepine. Asterisks denote significant paired t-tests (p<0.05) with effect sizes greater than 1, when compared to healthy (black asterisks) or MDD (grey asterisks). N=200 randomized microcircuits per condition (4.5 s-long duration per simulation) for panels

FIG. 6C is a line graph showing distributions of pre- and post-stimulus firing rates.

FIG. 6D is a bar graph showing probability of failed detection and false detection with non-selective PAM was worsened and unchanged, respectively, compared to the MDD condition. Conditions, from left to right in each set of four bars: healthy, MDD, MDD+α5-PAM, MDD+benzodiazepine. Asterisks denote significant paired t-tests (p<0.05) with effect sizes greater than 1, when compared to healthy (black asterisks) or MDD (grey asterisks). N=200 randomized microcircuits per condition (4.5 s-long duration per simulation) for panels

FIG. 6E is a line graph showing EEG PSD, bootstrapped mean, and 95% confidence intervals. Inset: EEG PSD in log scale.

FIG. 6F is a line graph showing spikes PSD of Pyr neurons, bootstrapped mean, and 95% confidence intervals. Inset: spikes PSD in log scale.

FIG. 6G is a line graph showing fitted aperiodic components of the EEG PSD for each condition. Upper inset: broadband (3-30 Hz) area under the curve (AUC or integral). Lower inset: exponent (χ). Asterisks denote significant paired t-tests (p<0.05) with effect sizes greater than 1, when compared to healthy (black asterisks) or MDD (grey asterisks). N=50 randomized microcircuits per condition (25 s-long duration per simulation).

FIG. 6H is a line graph showing fitted periodic component of the EEG PSD for each condition. Inset plots: integral of the power spectral density in the theta (θ) and beta (β) frequency ranges. Asterisks denote significant paired t-tests (p<0.05) with effect sizes greater than 1, when compared to healthy (black asterisks) or MDD (grey asterisks). N=50 randomized microcircuits per condition (25 s-long duration per simulation).

FIG. 7A illustrates a connectivity schematic highlighting the mechanisms of depression (MDD; loss of SST tonic and synaptic inhibition to all cells) and α5-PAM doses (boosted SST tonic and synaptic inhibition to Pyr neurons).

FIG. 7B is a schematic illustration of an experiment simulating five levels of SST loss severity (0%, 10%, 20%, 30%, 40%) across 20 different circuits, representing a total of 100 different virtual subjects.

FIG. 8A shows power spectral density (PSD) of simulated EEG from each severity level of SST loss (bootstrapped mean and 95% confidence intervals across circuits). Inset—PSD plotted in log scale.

FIG. 8B shows fitted aperiodic components (1/f) of the PSD for each condition (bootstrapped mean and 95% confidence intervals across circuits). Inset—1/f component plotted in log scale.

FIG. 8C shows PSD power in the alpha range (8-12 Hz) for each level of SST loss (grey—healthy standard deviation). All asterisks denote significant paired t-tests (p<0.05) with effect sizes greater than 1 when compared to healthy.

FIG. 9A shows exemplary power spectral density (PSD) profiles for an example circuit. Depicted are healthy range (dashed line), MDD—30% SST (dotted line), and 100% dose (solid line).

FIG. 9B illustrates dose-response for an example circuit with 30% SST loss (open circle upper right), plotted across three candidate EEG biomarkers of depression severity (1/f AUC, θ AUC, α AUC).

FIG. 9C illustrates predicted optimal doses for each circuit plotted against its EEG biomarkers in MDD before drug application.

FIG. 9D illustrates percent of correct dose prediction for test circuit sets using multivariate (MV) or single EEG biomarkers (1000 permutations; blue=under-estimated errors; red=over-estimated errors).

FIG. 9E illustrates distributions of pre-stimulus firing rates from an example circuit with 40% SST loss (n=23,950 windows of 50 ms, dashed line) before (magenta) and after (blue) predicted dose, and average healthy post-stimulus firing rates (n=200 windows of 50 ms, solid black line).

FIG. 9F illustrates mean and standard deviation of spike rate (left), failed detection rates (middle), and false detection rates (right) in simulated MDD circuits before and after applying the predicted drug dose.

FIG. 10A illustrates variance due to circuit connectivity randomizations was larger than the variance due to activity state randomizations (10 healthy circuit seeds across 10 activity state seeds). Error bars show SD.

FIG. 10B illustrates average SD of spike rate across circuits or states.

FIG. 10C illustrates dose response for an example circuit with severity 40% SST loss, which was used to calculate the optimal dose (diamonds) and optimal dose range (triangles) with respect to the healthy mean and ranges (shaded grey area).

FIG. 10D illustrates optimal dose (dots) and ranges (shaded area) for each circuit plotted as a function of its spike rate in MDD, before drug application.

FIG. 10E illustrates percent of correct prediction of dose for test circuits sets (1000 permutations).

FIG. 10F illustrates mean and SD of spike rates (left), failed detection rates (middle), and false detection rates (right) in simulated MDD circuits and after applying predicted doses.

FIG. 11A illustrates spike rate (Hz) of the PSD metric 1/f.

FIG. 11B illustrates spike rate (Hz) of the PSD metric, theta (θ).

FIG. 11C illustrates spike rate (Hz) of the PSD metric alpha (α).

FIG. 12A illustrates prediction of optimal dose (top) and resulting accuracy scores (bottom) using an ANN (either 3 or 1 input nodes, 9 hidden layer ReLU nodes, 1 output linear node).

FIG. 12B illustrates prediction of optimal dose (top) and resulting accuracy scores (bottom) using an SVM with a linear kernel.

FIG. 12C illustrates prediction of optimal dose (top) and resulting accuracy scores (bottom) using a sigmoidal regression.

### DETAILED DESCRIPTION

The present inventors have developed computational models that mechanistically link drug effects on brain microcircuitry to EEG signatures and provide EEG-based biomarkers useful for determining patient-specific therapeutic dosing and monitoring drug efficacy.

In a proof of concept study, described in more detail below, the effect of an α5-PAM on tonic inhibition recorded in human neurons was modeled followed by testing α5-PAM effects on cortical processing using detailed data-driven computational models of human cortical microcircuits in health and depression. The simulations show that α5-PAMs efficaciously and robustly recovered cortical processing back to healthy levels, as quantified by stimulus detection metrics. In addition, power spectral biomarkers for assessing drug efficacy were identified. By comparison, simulation of non-selective PAMs did not recover cortical function and had different EEG signatures.

The methods described here are useful to de-risk and facilitate clinical translation of PAMs, including α5-PAMs, by providing EEG-based biomarkers for therapeutic monitoring and dosing as well as an in silico framework for assessing drug pharmacology.

### GABA Receptor Positive Allosteric Modulators

GABA-positive allosteric modulators (PAM) are medications that target the GABA-A receptor. The GABA-A receptor consists of two α (alpha) subunits, two β (beta) subunits and one γ (gamma) subunit forming a pentameric protein that functions as a ligand-gated chloride channel. The natural ligand of the GABA-A receptor is gamma-aminobutyric acid (GABA), an inhibitory neurotransmitter. Ligand binding opens the channel, allowing chloride to flow into the cell and resulting in hyperpolarization and a diminished action potential, which prevents the release of excitatory neurotransmitters. PAMs exert their action by increasing agonist effects, for example by increasing the frequency with which the chlorine channel opens when an agonist binds the GABA receptor. Exemplary PAMs include benzodiazepines, barbiturates, and induction anesthetics such as propofol, etomidate, and ketamine. An exemplary α5-PAM is a benzodiazepine derivative such as GL-II-73, or a derivative thereof.

Numerous isoforms of GABAA receptors (also refereed to interchangeably as GABA-A or GABA_{A}) exist, where the isoforms are defined by their particular complement of α, β, and γ subunits. In humans, there are 6 different α subunits, 3 different β subunits, and 3 different γ subunits. Specific isoforms may be designated according to their subunit complement, for example “α5β3γ2S”, which refers to an α5 isoform located in hippocampal pyramidal cells.

### Electroencephalography

The methods described here utilize data obtained from electroencephalography (EEG), which is a neurophysiological technique for recording electrical activity of the brain. Brain activity is measured as a function of time varying potentials via electrodes placed in a standard pattern on the scalp. The methods described here may utilize stored or transmitted EEG data. In this context, the EEG data may be stored on computer-readable media or transmitted over a network. For use in the methods described here, the EEG data is preferably recorded using electrodes placed in accordance with an accepted standard, such as the International 10/20 placement system. A multi-channel recording of brain activity produces raw data which is digitized and processed, for example, using a Fast Fourier Transform (FFT) or related signal processing methods. In accordance with the methods described here, the data is processed to obtain power spectral density.

Computation of the power spectrum typically includes segmenting the continuous signal, applying Fourier analysis to each segment, and calculating the mean over segments of the power at each frequency. Segment length can vary, but segments are typically at least about 1-5 seconds in duration. In accordance with the methods described here, EEG analysis may be focused in the delta (0.5-4 Hz), theta (4-7 Hz), low alpha (8-10 Hz), high alpha (10-12 Hz), beta (13-30 Hz), and/or gamma (30-40 Hz) frequency bands.

Various parameters may be derived from the power spectrum including power spectral density (PSD) as discussed herein. PSD represents the power distribution of EEG series in the frequency domain. The relative PSD for a sub-band may be obtained by dividing the PSD of each frequency band by the total PSD of the whole frequency band estimated according to methods known in the art. PSD was calculated using Welch's method, with a 3 s Hanning window and 30% window overlap. For detail, see Guet-McCreight et al., 2023 bioRxiv and Mazza et al., 2023 PLOS Computational Biology 19 (4), e1010986.

### Methods of Treating

The disclosure provides methods for determining a therapeutic dose and monitoring therapeutic efficacy of a PAM based on EEG data obtained from the patient. The EEG data is utilized to obtain power spectral density (PSD) information which is in turn inputted into a dose prediction, which is utilized to determine the therapeutic dose and/or efficacy for the subject. In aspects, the dose prediction function is derived in silico from virtual subjects of different depression severity. In aspects, the dose prediction function also can be applied to a pre-treatment condition and then to a post-treatment condition of the subject to calibrate and shorten the dosing process.

In aspects, provided are methods of treating a subject in need thereof with a GABAA-positive allosteric modulator (PAM), the methods comprising determining a therapeutic dose of the PAM for the subject by a process including obtaining or receiving an electroencephalography (EEG) signal from the subject, performing, on a processor, a power spectral density (PSD) calculation on the signal to obtain a raw PSD, decomposing, on a processor, the raw PSD into its aperiodic and periodic components, and determining, based on the raw PSD, the aperiodic and periodic components, and a dose prediction function, the therapeutic dose of the PAM for the subject.

Also provided are methods of monitoring therapeutic efficacy of a GABAA-positive allosteric modulator (PAM) in a subject in need thereof, the methods including obtaining or receiving an electroencephalography (EEG) signal from the subject in a pre-treatment condition and at least one subsequent post-treatment condition, where the pre-treatment condition corresponds to pre-treatment with the PAM and the post-treatment condition corresponds to after administration of at least one dose of the PAM to the subject, performing, on a processor, a power spectral density (PSD) calculation on the signal obtained or received for each condition to obtain raw PSD for each condition, decomposing, on a processor, the raw PSD into its aperiodic and periodic components for each condition, determining, based on the raw PSD and the aperiodic, periodic components, and a dose prediction function derived in silico from virtual subjects of different depression severity, the dose prediction for the subject for each condition; comparing the predicted dose at each condition, where, a decrease in predicted dose indicates that the PAM is having a therapeutic effect, and, optionally, administering a second or further dose of the PAM to the subject and repeating until the predicted dose is either 0 or smaller than a predetermined level.

In an aspect, the disclosure provides methods of treating a subject in need thereof with an α5-PAM, the method comprising determining a therapeutic dose of the α5-PAM for the subject by a process including obtaining or receiving an electroencephalography (EEG) signal from the subject, performing, on a processor, a power spectral density (PSD) calculation on the signal to obtain a raw PSD, decomposing, on a processor, the raw PSD into its aperiodic and periodic components, and determining, based on the raw PSD, the aperiodic, periodic components, and a dose prediction function derived in-silico from virtual subjects of different depression severity, the therapeutic dose of the α5-PAM for the subject.

In an aspect, the disclosure also provides methods of monitoring therapeutic efficacy of an α5-PAM in a subject in need thereof, the methods including obtaining or receiving an electroencephalography (EEG) signal from the subject in a pre-treatment condition and at least one subsequent post-treatment condition, where the pre-treatment condition corresponds to pre-treatment with an α5-PAM and the post-treatment condition corresponds to after administration of at least one dose of the α5-PAM to the subject, performing, on a processor, a power spectral density (PSD) calculation on the signal obtained or received for each condition to obtain raw PSD for each condition, decomposing, on a processor, the raw PSD into its aperiodic and periodic components for each condition, and determining, based on the raw PSD, the aperiodic and periodic components, and a dose prediction function derived in silico from virtual subjects of different depression severity, the dose prediction for the subject for each condition; comparing the predicted dose at each condition, where, a decrease in predicted dose from the previous to the subsequent condition indicates that the PAM is having a therapeutic effect, and, optionally, administering a second or further dose of the PAM to the subject and repeating until the predicted dose is 0 (or smaller than a predetermined level).

The disclosure also provides methods for determining a therapeutic dose and monitoring therapeutic efficacy of a GABAA-positive allosteric modulators (PAM) based on EEG data obtained from the patient. The EEG data is utilized to obtain power spectral density (PSD) information which is in turn utilized to estimate the level of PSD deviation from healthy (“PSDD”) for the subject, and determining, based on the subject's PSDD level, the therapeutic dose and/or efficacy for the subject. As discussed in more detail infra, estimating the subject's PSDD may comprise comparing one or more of the raw PSD and the aperiodic and/or periodic components to a reference, which may be, for example, an average across healthy models, an average obtained from EEG measurements of healthy subjects, or a predetermined healthy PSD.

In aspects, provided are methods of treating a subject in need thereof with a GABAA-positive allosteric modulators (PAM), the methods comprising determining a therapeutic dose of the PAM for the subject by a process including obtaining or receiving an electroencephalography (EEG) signal from the subject, performing, on a processor, a power spectral density (PSD) calculation on the signal to obtain a raw PSD, decomposing, on a processor, the raw PSD into its aperiodic and periodic components, estimating, based on the raw PSD and the aperiodic and periodic components, a level of PSD deviation from healthy (“PSDD”) for the subject, and determining, based on the subject's PSDD level, the therapeutic dose of the PAM for the subject.

Also provided are methods of monitoring therapeutic efficacy of a GABAA-positive allosteric modulators (PAM) in a subject in need thereof, the methods including obtaining or receiving an electroencephalography (EEG) signal from the subject in a pre-treatment condition and at least one subsequent post-treatment condition, where the pre-treatment condition corresponds to pre-treatment with the PAM and the post-treatment condition corresponds to after administration of at least one dose of the PAM to the subject, performing, on a processor, a power spectral density (PSD) calculation on the signal obtained or received for each condition to obtain raw PSD for each condition, decomposing, on a processor, the raw PSD into its aperiodic and periodic components for each condition, estimating, based on the raw PSD and the aperiodic and periodic components, a level of PSD deviation from healthy (“PSDD”) for the subject for each condition, comparing the subject's PSDD level at each condition, where, a decrease in PSDD indicates that the PAM is having a therapeutic effect, and, optionally, administering a second or further dose of the PAM to the subject and repeating until the subject's PSDD level has reached a predetermined level.

In an aspect, the disclosure provides methods of treating a subject in need thereof with an α5-PAM, the method comprising determining a therapeutic dose of the α5-PAM for the subject by a process including obtaining or receiving an electroencephalography (EEG) signal from the subject, performing, on a processor, a power spectral density (PSD) calculation on the signal to obtain a raw PSD, decomposing, on a processor, the raw PSD into its aperiodic and periodic components, estimating, based on the raw PSD and the aperiodic and periodic components, a level of PSD deviation from healthy (“PSDD”) for the subject, and determining, based on the subject's PSDD level, the therapeutic dose of the α5-PAM for the subject.

In an aspect, the disclosure also provides methods of monitoring therapeutic efficacy of an α5-PAM in a subject in need thereof, the methods including obtaining or receiving an electroencephalography (EEG) signal from the subject in a pre-treatment condition and at least one subsequent post-treatment condition, where the pre-treatment condition corresponds to pre-treatment with an α5-PAM and the post-treatment condition corresponds to after administration of at least one dose of the α5-PAM to the subject, performing, on a processor, a power spectral density (PSD) calculation on the signal obtained or received for each condition to obtain raw PSD for each condition, decomposing, on a processor, the raw PSD into its aperiodic and periodic components for each condition, estimating, based on the raw PSD and the aperiodic and periodic components, a level of PSD deviation from healthy (“PSDD”) for the subject for each condition, comparing the subject's PSDD level at each condition, where, a decrease in PSDD indicates that the α5-PAM is having a therapeutic effect, and, optionally, administering a second or further dose of the α5-PAM to the subject and repeating until the subject's PSDD level has reached a predetermined level.

In accordance with the foregoing aspects, the methods may also include where the aperiodic components comprise broadband AUC and exponent (χ). In aspects, the methods may also include where the raw PSD and periodic components comprise the power measured by integral of one or more PSD frequency bands selected from theta, alpha, and beta frequency bands, and/or sub-bands thereof. In aspects, the methods may also include where estimating the subject's PSDD level includes determining a ratio of one or more of the raw PSD and the aperiodic and/or periodic components. In aspects, the ratio is a ratio of a PSD for a pre-treatment condition and a post-treatment condition of the subject in at least one of theta (4-8 Hz), alpha (8-12 Hz), beta (12-30 Hz) and gamma (30-100 Hz) frequency bands, and/or one or more sub-bands thereof. In aspects, the ratio is a ratio of broadband AUC or exponent (χ) for a pre-treatment condition and a post-treatment condition of the subject. In aspects, the methods may also include where estimating the subject's PSDD includes comparing one or more of the raw PSD and the aperiodic and/or periodic components to a reference. In aspects, the reference is an average across healthy models, an average obtained from EEG measurements of healthy subjects, a predetermined healthy PSD (which may have been determined from computational models and/or EEG measurements of healthy subjects). In aspects, the reference is a reference frequency band, or ratio. In aspects, the methods may also include where the ratio is a ratio of a PSD of the reference to the corresponding PSD of the subject in at least one of theta (4-8 Hz), alpha (8-12 Hz), beta (12-30 Hz) and gamma (30-100 Hz) frequency bands, and/or one or more sub-bands thereof. In aspects, the methods may also include where the ratio is a ratio of a PSD of the reference to the corresponding PSD of the subject in at least one of broadband AUC or exponent (χ).

In aspects of any of the foregoing methods, the subject in need of treatment may be one diagnosed with a neuropsychiatric disorder, a neurological disorder, a neurodegenerative disorder, or any combination thereof. In aspects, the neuropsychiatric disorder is bipolar disorder or schizophrenia. In aspects, the neurological disorder is epilepsy or other brain related disorders. In aspects, the neurodegenerative disorder is Alzheimer's disease or dementia.

In aspects of any of the foregoing methods, the subject in need includes one who has been diagnosed with depression. In aspects, the subject has been diagnosed with major depressive disorder (MDD). In aspects, the depression or MDD is treatment resistant.

In accordance with any of the foregoing methods, the subject is preferably a human subject.

In accordance with aspects of the methods described here, dose prediction is for a pre-treatment condition and a post-treatment condition of the subject.

In accordance with aspects of the methods described here, PSD metrics of a subject may comprise one or more of the raw PSD and the aperiodic and/or periodic components in at least one of theta (4-8 Hz), alpha (8-12 Hz), beta (12-30 Hz) and gamma (30-100 Hz) frequency bands, and/or one or more sub-bands thereof. In aspects, the methods may also include PSD metrics of the subject in at least one of broadband AUC or exponent ( ).

In accordance with aspects of the methods described here, estimating the subject's PSDD level may comprise determining a ratio of one or more of the raw PSD and the aperiodic and/or periodic components. In aspects, the ratio is a ratio of a PSD for a pre-treatment condition and a post-treatment condition of the subject in at least one of theta (4-8 Hz), alpha (8-12 Hz), beta (12-30 Hz) and gamma (30-100 Hz) frequency bands, and/or one or more sub-bands thereof. In aspects, the ratio is a ratio of broadband AUC or exponent (χ) for a pre-treatment condition and a post-treatment condition of the subject.

In accordance with the methods described here, estimating the subject's PSDD may comprise comparing one or more of the raw PSD and the aperiodic and/or periodic components to a reference. In aspects, the reference is an average across healthy models, an average obtained from EEG measurements of healthy subjects, a predetermined healthy PSD (which may have been determined from computational models and/or EEG measurements of healthy subjects). In aspects, the reference is a reference frequency band, or ratio. In aspects, the methods may also include where the ratio is a ratio of a PSD of the reference to the corresponding PSD of the subject in at least one of theta (4-8 Hz), alpha (8-12 Hz), beta (12-30 Hz) and gamma (30-100 Hz) frequency bands, and/or one or more sub-bands thereof. In aspects, the methods may also include where the ratio is a ratio of a PSD of the reference to the corresponding PSD of the subject in at least one of broadband AUC or exponent (χ).

In aspects of any of the foregoing methods, the EEG signal is obtained over a period of time. The period of time may be from 2-5 minutes, from 5-15 minutes, from 15-30 minutes, from 30-60 minutes, or from 60-120 minutes. In aspects, the period of time may be about 5, 10, 15, 20, 25, 30, 35, 40, 45, 50, 60, 90, or 120 minutes.

In aspects of any of the foregoing methods, the α5-PAM is benzodiazepine derivative such as GL-II-73, or a derivative thereof.

The present invention is further described in the following examples that are intended for illustration purposes only, since numerous modifications and variations will be apparent to those skilled in the art. The examples demonstrate the utility of the methods described here for determining a therapeutic dose of a PAM and monitoring therapeutic efficacy based on a power spectral analysis of EEG data obtained from a subject. Described are proof of concept studies testing α5-PAM effects on cortical processing using detailed data-driven computational models of human cortical microcircuits in health and depression. The simulations show that α5-PAMs efficaciously and robustly recovered cortical processing back to healthy levels, as quantified by stimulus detection metrics and identified power spectral biomarkers for assessing drug efficacy.

### Proof of Concept Studies

The effects of α5-PAM on cortical microcircuits was evaluated by first modeling the α5-PAM modulation of tonic inhibition currents as recorded in single human neurons and then integrating the α5-PAM effect into two biophysically detailed human L2/3 microcircuit models of healthy and major depression disorder (MDD) conditions. Both models include key neuron types (Pyr, PV, SST, and VIP neurons). The MDD microcircuit model includes a 40% reduction in SST interneuron tonic and synaptic inhibition onto the other neurons in the microcircuit. These two models are discussed in detail in Yao et al. 2022 Cell Reports January 11; 38(2):110232. “Reduced inhibition in depression impairs stimulus processing in human cortical microcircuits”; and Mazza et al. 2023 PLOS Computational Biology 19 (4), e1010986 “In silico EEG biomarkers of reduced inhibition in human cortical microcircuits in depression”, the contents of each of which are hereby incorporated by reference in their entireties.

Briefly, both models consist of 1000 neurons distributed in a 500×500×950 μm³ volume and include the four key neuron types in cortical L2/3: Pyramidal (Pyr), Somatostatin-expressing (SST), Parvalbumin-expressing (PV), and Vasoactive Intestinal Peptide-expressing (VIP) neurons. The proportions of the neuron types were: 80% Pyr, 5% SST, 7% PV, and 8% VIP in accordance with relative L2/3 neuron densities and RNA-seq data. The models were simulated using NEURON (Carnevale et al., The NEURON Book: Cambridge University Press; 2006) and LFPy (Hagen et al., Multimodal modeling of neural network activity: computing LFP, ECoG, EEG and MEG signals with LFPy2.0. bioRxiv. 2018; 281717. doi:10.1101/281717). The neuronal morphology reconstructions of the multi-compartment models were obtained from the Allen Cell Types database and the models were fitted using multi-objective optimization (Hay et al., 2011; Van Geit et al., 2016) with either single cell data from the Allen Brain Institute (putative PV, SST, and VIP inhibitory neuron fits; Gouwens et al., 2018) or population data from the Krembil Brain Institute (Pyr neuron fits; Chameh et al., 2021). Synaptic parameters in these models were fit to human data where possible (Komlósi et al., 2012; Obermayer et al., 2018; Seeman et al., 2018; Szegedi et al., 2016) and to curated rodent data otherwise (Ramaswamy et al., 2015). While there are other connection types in the circuit models, the prototypical connection types are described here: the Pyr→Pyr excitatory synapses were placed on both basal and apical dendritic compartments, the PV→Pyr inhibitory connections were placed on basal dendritic compartments, the SST→Pyr inhibitory connections were placed on apical dendritic compartments, and the VIP inhibitory connections were placed on other inhibitory interneuron dendrites. These models are also constrained to replicate in vivo baseline and response spike rates for different neuron types. Depression microcircuits were modelled by reducing the conductance of SST interneuron synaptic and tonic inhibition on all cell types by 40%. For Pyr neurons in the depression model, tonic inhibition conductance was reduced by 40% on only apical dendritic compartments. For each interneuron type in the depression model, the contributions of SST interneurons to tonic inhibition were estimated and this contribution was reduced by 40%.

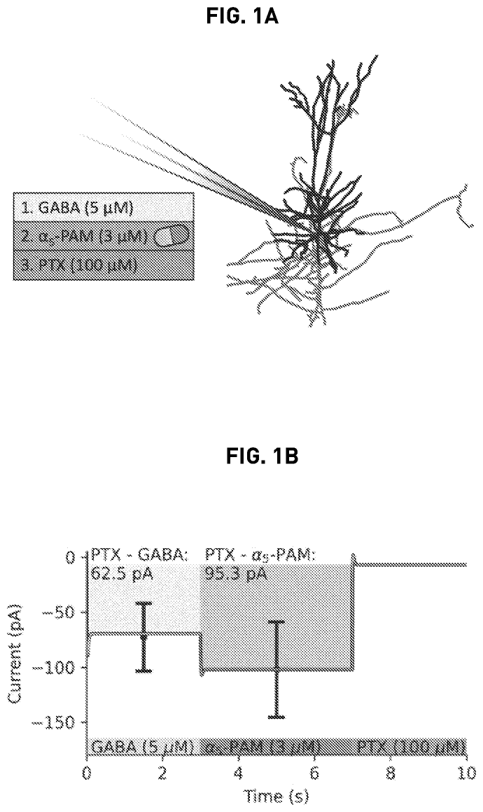

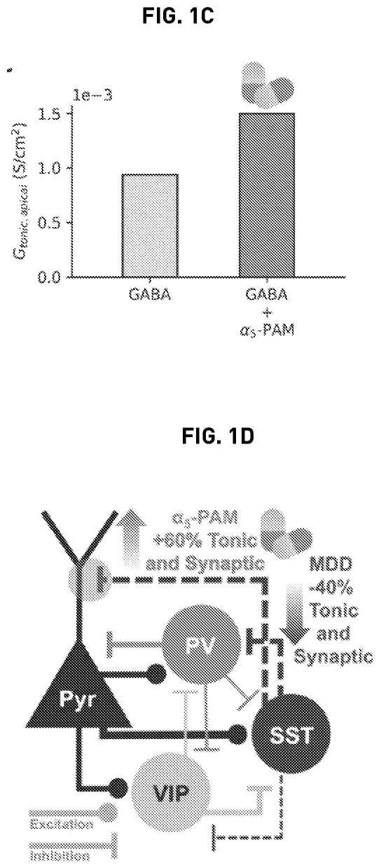

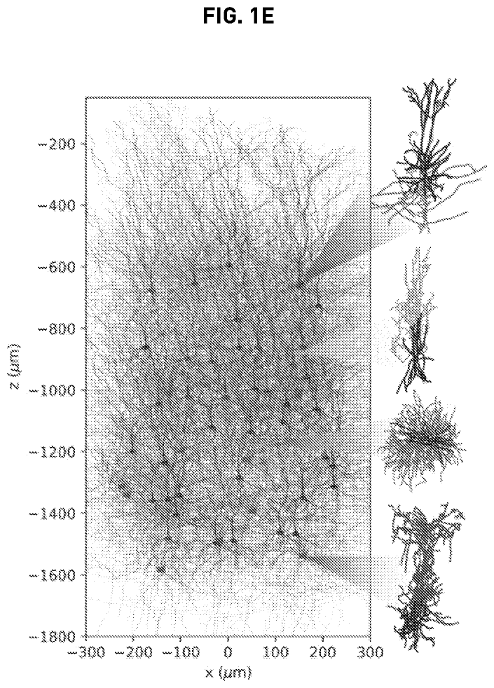

FIG. 1 illustrates the effects of α5-PAM on tonic inhibition modeled in Pyr neurons by constraining a model of human L2/3 Pyr neurons to reproduce the average currents recorded in human neurons in vitro. To do this, the tonic inhibition conductance (G_{tonic}) was fitted in the neurons (uniformly in soma, basal dendrites, and apical dendrites) to obtain the current magnitude recorded when applying GABA (FIG. 1A, FIG. 1B, FIG. 1C). Then, the α5-PAM modulation of G_{tonic} was fitted in the apical dendrites to reproduce the 52% increase in current magnitude during application of a therapeutically relevant dose of α5-PAM in addition to GABA (−65.9±30.8 pA vs −95.3±43.6 pA, paired-sample t-test, p<0.05, Cohen's d=#, and estimated the α5-PAM modulation to be 60%. This modulation was integrated into models of human cortical microcircuits. In addition, it was applied to synaptic inhibition mediated by SST interneurons onto Pyr neuron apical dendrites (FIG. 1D), since these connection types in cortex are also α5-mediated. The detailed microcircuit models, with model neuron morphologies, are illustrated in FIG. 1E.

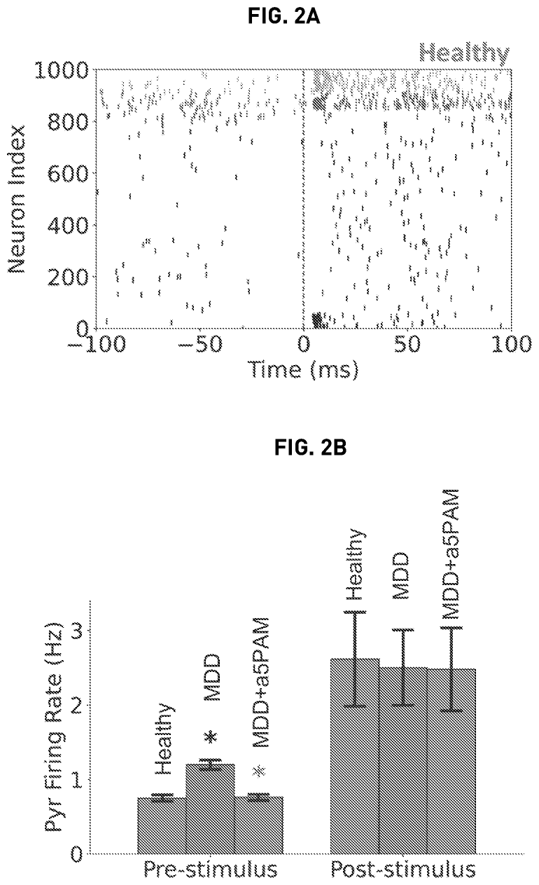

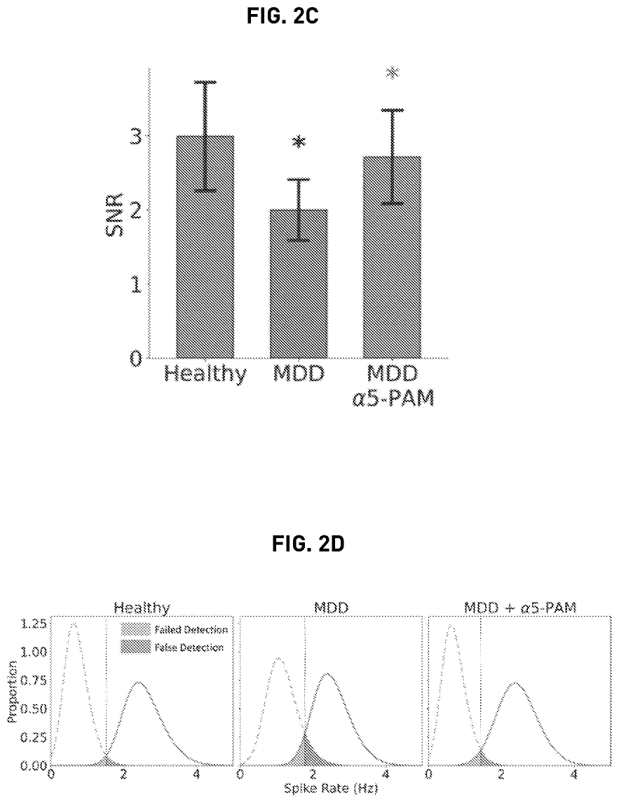

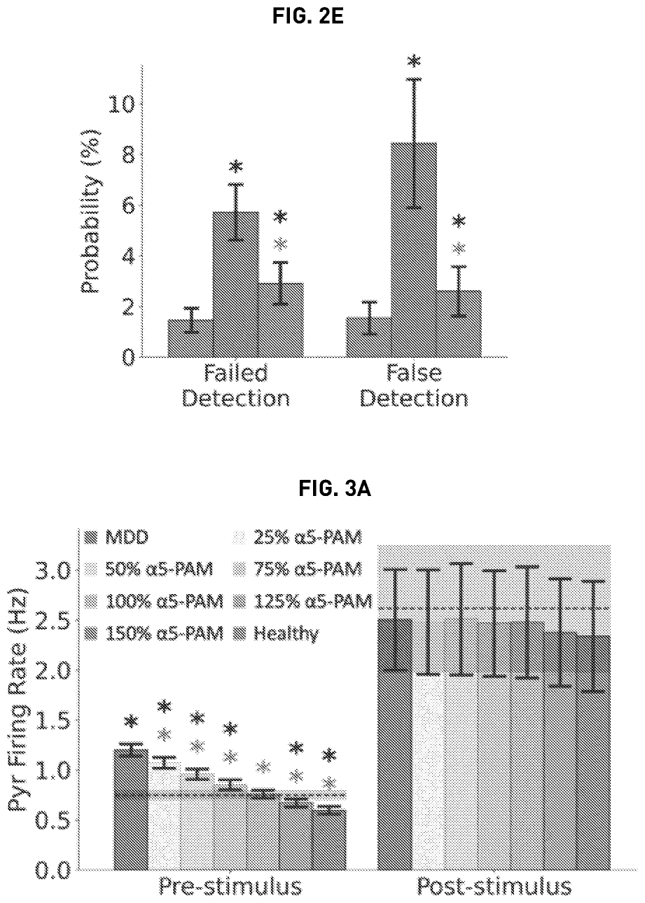

Next, the α5-PAM was tested on the human cortical microcircuits by simulating microcircuit baseline and response to brief stimuli in health, MDD, and MDD+α5-PAM. As previously demonstrated, compared to healthy circuits, reduced SST interneuron inhibition in MDD microcircuits resulted in increased baseline spike rates (FIG. 2A, FIG. 2B; Healthy: 0.75±0.04 Hz; MDD: 1.20±0.06 Hz; paired-sample t-test, p<0.05, Cohen's d=8.6), decreased signal to noise ratio (SNR) (FIG. 2C; Healthy: 2.99±0.73; MDD: 2.00±0.41; paired sample t-test, p<0.05, Cohen's d=−1.7), and worsened microcircuit function in terms of failed and false stimulus detection rates (FIG. 2D, FIG. 2E; Healthy: 1.45±0.47% and 1.54±0.63%, MDD: 5.71±1.10% and 8.44±2.53%, paired-sample t-test, p<0.05, Cohen's d=−5.0 & −3.7 respectively). Modulation of inhibition by a therapeutic dose of α5-PAM in simulated MDD microcircuits restored baseline spike rates (0.76±0.04 Hz, Cohen's d=0.2) and consequently SNR (2.71±0.63, Cohen's d=−0.4) back to healthy levels. The probability of failed and false stimulus detection errors was calculated based on the distribution of Pyr neuron firing at baseline vs response, averaged over 50 ms windows (FIG. 2D). The simulated therapeutic dose of α5-PAM recovered failed and false stimulus detection rates nearly back to healthy level (2.91±0.83% & 2.59±0.97%, Cohen's d=2.2 and 1.3).

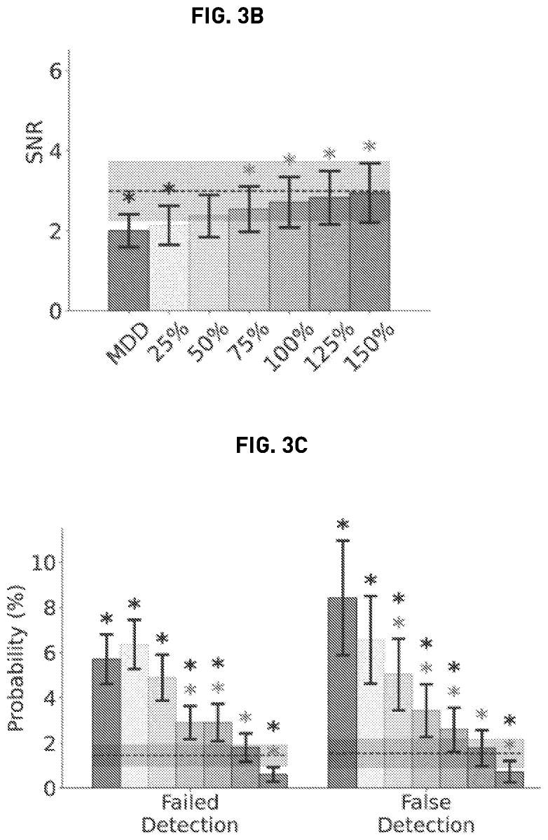

To test the effectiveness of higher or lower α5-PAM doses compared to the therapeutic dose at recovering microcircuit function, α5-PAM modulation of apical inhibition ranging 15-90%, corresponding to 25-150% of the estimated 60% was simulated (FIG. 3A). The therapeutic dose of α5-PAM (3 μM, corresponding to 100%) was the optimal dose that recovered baseline spike rates back to healthy levels (Healthy: 0.75±0.04 Hz; 100% α5-PAM: 0.76±0.04 Hz, Cohen's d=0.2), whereas lower doses were not sufficient (25% α5-PAM: 1.07±0.05 Hz, paired-sample t-test, p<0.05, Cohen's d=6.5) and higher doses over-reduced the spike rate (150% α5-PAM: 0.60±0.04 Hz, paired-sample t-test, p<0.05, Cohen's d=−3.8; FIG. 3A). The relationship between dose and effect on baseline rates was linear. There was a similar linear relationship between dose and SNR, although several doses were capable at restoring SNR back to healthy levels (100%-150%; FIG. 3B; Healthy: 2.99±0.73; 100% α5-PAM: 2.71±0.63, Cohen's d=−0.4; 125% α5-PAM: 2.82±0.66, Cohen's d=−0.2; 150% α5-PAM: 2.95±0.74, Cohen's d=−0.1). For doses greater than 100% the SNR was preserved because both baseline and response rates were similarly dampened, which was not as optimal as the 100% effect. The relationship between dose and failed and false stimulus detection errors was non-linear, with a poor effect for low doses (25-50%; 25% α5-PAM: 6.36±1.09% and 6.57±1.95%, paired-sample t-test, p<0.05, Cohen's d=5.8 & 3.5, compared to healthy) followed by a jump in recovery at 75% dose, so that for doses of 75-125% the error rates recovered close to healthy levels (FIG. 3C; 100% α5-PAM: 2.91±0.83% & 2.59±0.97%, paired-sample t-test, p<0.05, Cohen's d=2.2 and 1.3, compared to healthy). Higher doses (150%) further reduced the failed and false error rates even below the healthy level (150% α5-PAM: 0.61±0.32% and 0.73±0.48%, paired-sample t-test, p<0.05, Cohen's d=−2.1 and −1.4, compared to healthy), possibly through a greater dampening of baseline activity compared to response when compared to healthy (Cohen's d=−3.8 and −0.5, respectively).

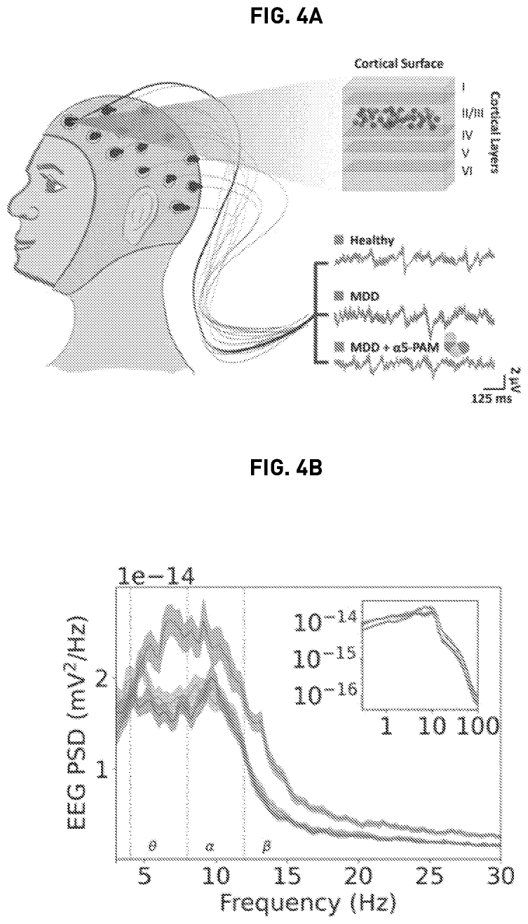

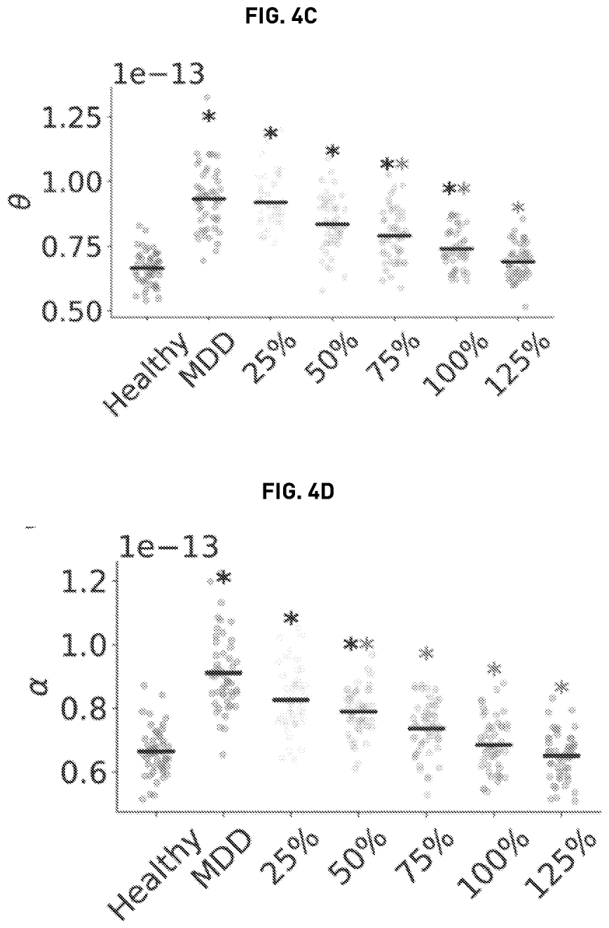

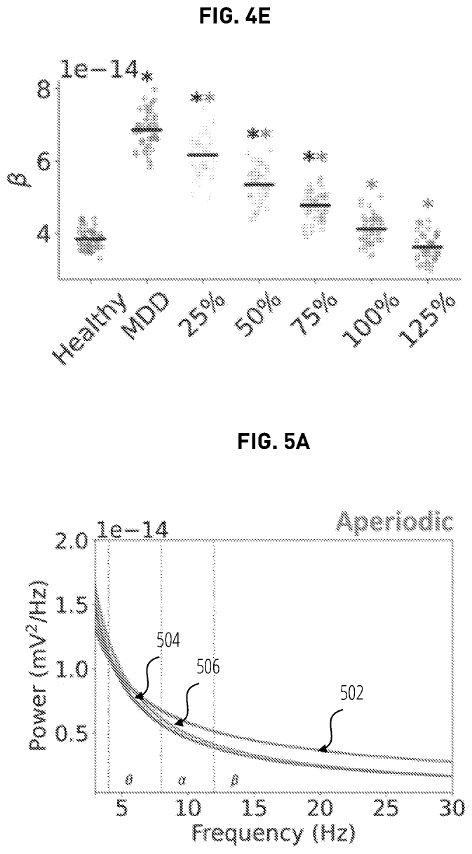

Next, EEG was simulated together with the microcircuit activity in the different conditions to identify signatures of α5-PAM efficacy in a clinically-relevant non-invasive signal (FIG. 4A). Simulated EEG signals generated by the MDD microcircuit models with reduced SST interneuron inhibition exhibited varying increased power in theta (Healthy: 6.65×10⁻¹⁴±6.34×10⁻¹⁵ mV²; MDD: 9.34×10⁻¹⁴±1.20×10⁻¹⁴ mV²; paired-sample t-test, p<0.05, Cohen's d=2.8), alpha (Healthy: 6.65×10⁻¹⁴±7.32×10⁻¹⁵ mV²; MDD: 9.12×10⁻¹⁴±1.15×10⁻¹⁴ mV²; paired-sample t-test, p<0.05, Cohen's d=2.5), and beta (Healthy: 3.84×10⁻¹⁴±2.78×10⁻¹⁵ mV²; MDD: 6.86×10⁻¹⁴±4.99×10⁻¹⁵ mV²; paired-sample t-test, p<0.05, Cohen's d=7.4) frequency bands (FIG. 4B). When α5-PAM was applied to the microcircuits at the therapeutic dose, the power spectral density profile was restored close to the healthy at all frequency bands, except for a slight shift in theta band peak (100% α5-PAM compared to healthy −θ: 7.41×10⁻¹⁴±7.71×10⁻¹⁵ mV², Cohen's d=1.1; α: 6.85×10⁻¹⁴±8.44×10⁻¹⁵ mV², Cohen's d=0.2; β: 4.12×10⁻¹⁴±3.78×10⁻¹⁵ mV², Cohen's d=0.8). There was a linear relationship between the effect of different α5-PAM doses at restoring power in theta (FIG. 4C), alpha (FIG. 4D), and beta (FIG. 4E) frequency bands. The therapeutic dose was necessary to sufficiently restore the power profile in alpha and beta bands, but for theta band a slightly higher dose was required to fully restore the power profile (125% α5-PAM compared to healthy −θ: 6.90×10⁻¹⁴±6.60×10⁻¹⁵ mV², Cohen's d=0.4).

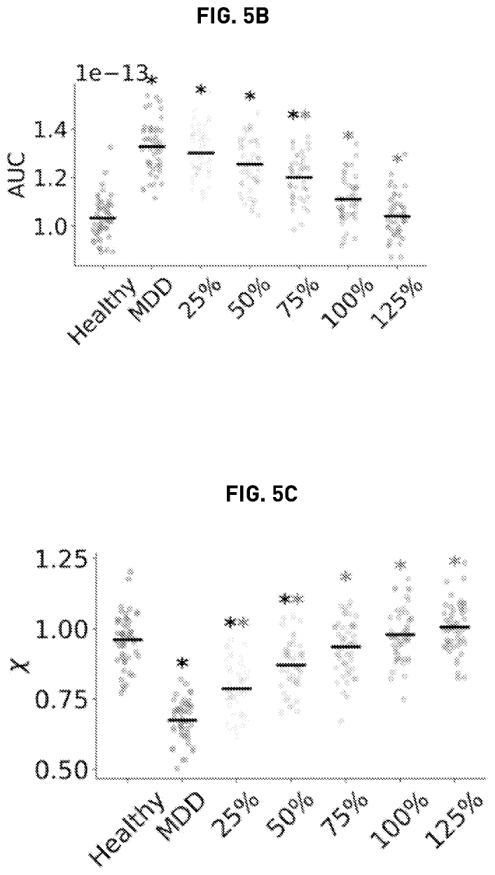

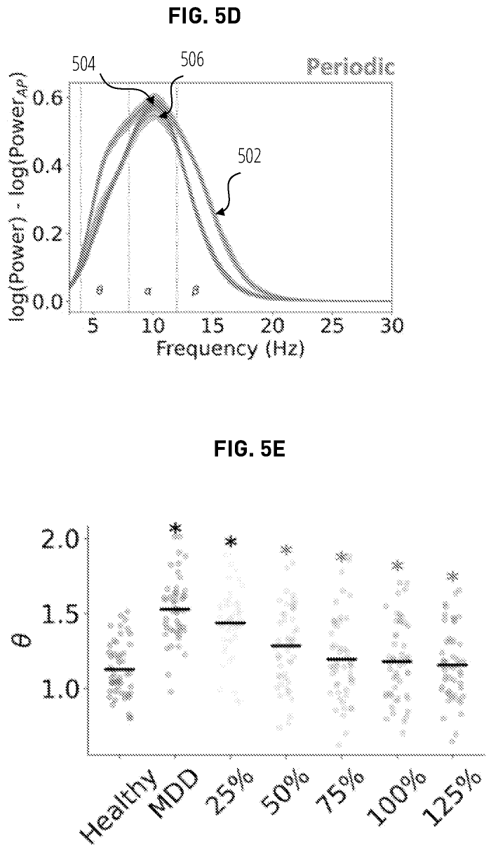

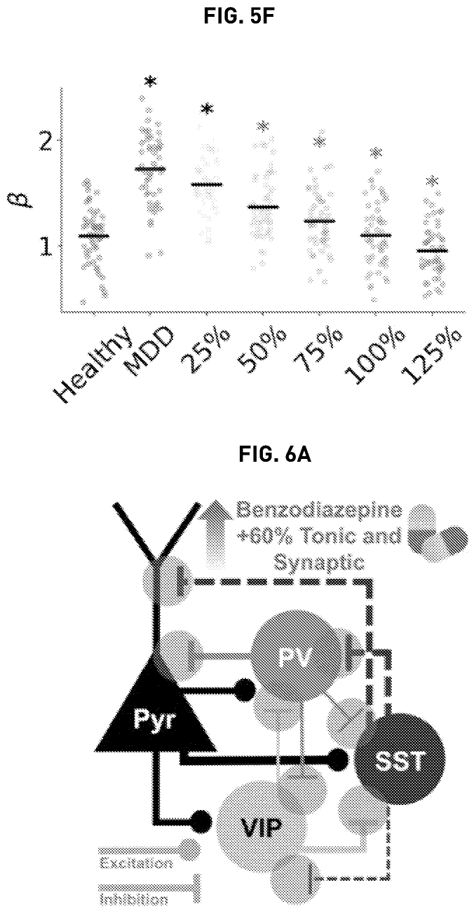

To further assess features of the spectral biomarkers of α5-PAM efficacy, the power spectral density profiles were decomposed into aperiodic (FIG. 5A, FIG. 5B) and periodic (FIG. 5D, FIG. 5E, FIG. 5F) components. MDD microcircuits with reduced SST interneuron inhibition primarily exhibited altered aperiodic exponents (Healthy: 0.96±0.09 mV²/Hz; MDD: 0.67±0.07 mV²/Hz; paired-sample t-test, p<0.05, Cohen's d=−3.4) and broadband power (Healthy: 1.03×10⁻¹³±8.49×10⁻¹⁵ mV²; MDD: 1.33×10⁻¹³±1.04×10⁻¹⁴ mV²; paired-sample t-test, p<0.05, Cohen's d=3.1) as well as increased periodic theta (Healthy: 1.12±0.18 mV²; MDD: 1.53±0.21 mV²; paired-sample t-test, p<0.05, Cohen's d=2.0) and lower beta (Healthy: 1.09±0.28 mV²; MDD: 1.73±0.33 mV²; paired-sample t-test, p<0.05, Cohen's d=2.0) frequency range powers. The therapeutic α5-PAM dose (100%) was necessary to restore the aperiodic power (100% α5-PAM compared to healthy: 1.11×10⁻¹³±9.84×10⁻¹⁵ mV², Cohen's d=0.8) and exponent (100% α5-PAM compared to healthy: 0.98±0.09 mV²/Hz, Cohen's d=0.2), as well as the periodic power in theta (100% α5-PAM compared to healthy: 1.18±0.26 mV², Cohen's d=0.2) and beta (100% α5-PAM compared to healthy: 1.10±0.30 mV², Cohen's d=0.03) bands back to the healthy levels.

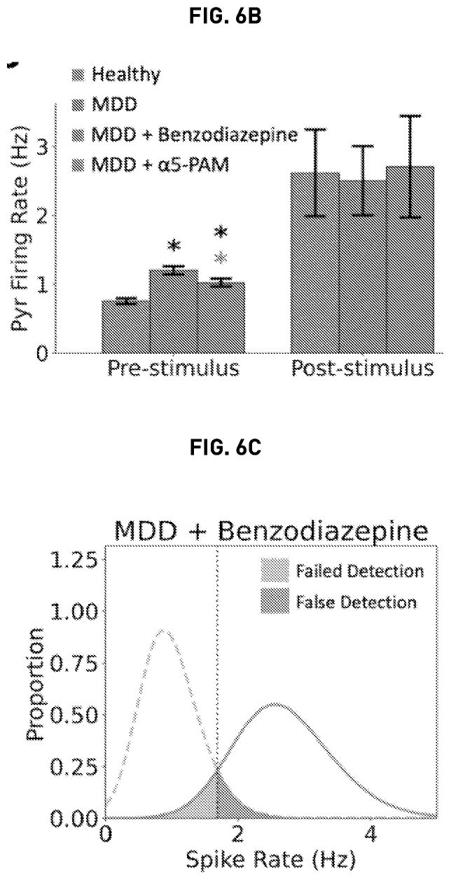

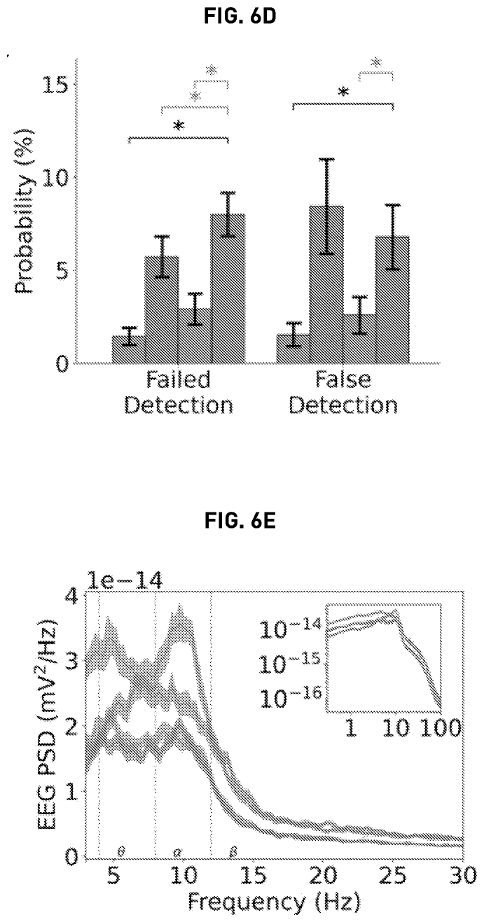

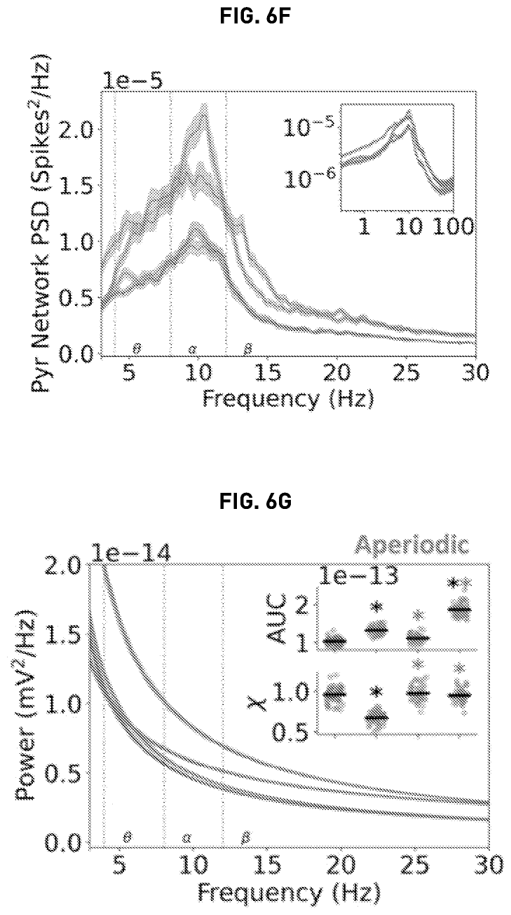

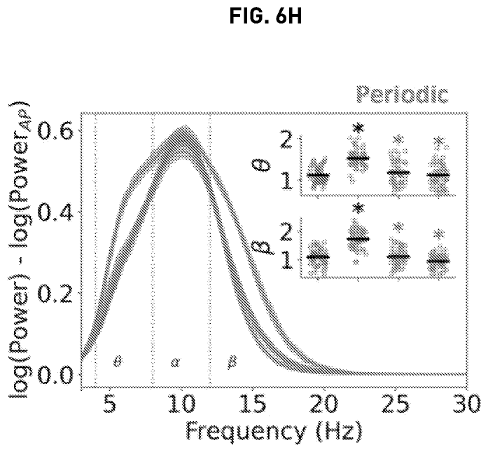

Next, the efficacy and EEG biomarkers of simulated α5-PAM with those of non-selective PAM, e.g. corresponding to benzodiazepine, were compared by simulating a 60% increase in tonic and synaptic inhibition in all of the microcircuit inhibitory connections (FIG. 6A). Though simulated non-selective PAM reduced baseline microcircuit spike rates, it did not restore spike rates sufficiently back to healthy levels (FIG. 6B; non-selective PAM compared to healthy: 1.02±0.06 Hz, paired-sample t-test, p<0.05, Cohen's d=5.5). Accordingly, simulated non-selective PAM did not improve the failed and false detection rates (FIG. 6C, FIG. 6D; non-selective PAM: 7.99±1.17% and 6.78±1.73%, healthy: 1.45±0.47% and 1.54±0.63%, paired-sample t-test, p<0.05, Cohen's d=7.3 and 4.0).

To better understand the circuit dynamics generated by application of non-selective PAM, the simulated EEG power spectral density profile was analyzed (FIG. 6H), which exhibited increases in all frequency bands (FIG. 6G). Increased broadband power was similarly seen at the PSD of the Pyr neuron spiking (FIG. 6F). When decomposed into aperiodic and periodic components non-selective PAM caused a broadband upward shift in the aperiodic component (FIG. 6G, upper inset; healthy: 1.03×10⁻¹³±8.49×10⁻¹⁵ mV²; non-selective PAM: 1.86×10⁻¹³±1.61×10⁻¹⁴ mV²; paired-sample t-test, p<0.05, Cohen's d=6.4), but were sufficient in recovering the aperiodic exponent parameter (FIG. 6G, lower inset; healthy: 0.96±0.09 mV²/Hz; non-selective PAM: 0.95±0.08 mV²/Hz; Cohen's d=−0.1) as well as the periodic theta (healthy: 1.12±0.18 mV²; non-selective PAM: 1.12±0.27 mV²; Cohen's d=−0.01) and lower beta (healthy: 1.09±0.28 mV²; non-selective PAM: 0.94±0.23 mV²; Cohen's d=−0.6) magnitudes (FIG. 6H).

### Simulating EEG of Inhibition Loss Severity in Depression and Treatment Response

Described below is the demonstration of the in silico prediction of dose based on depression severity of SST loss as determined from a patient's EEG. The results show the prediction function and its performance on test in silico data, as well the comparison of performance using alternative and more sophisticated prediction tools such as artificial neural networks. The prediction function can be readily tested on preclinical/clinical data.

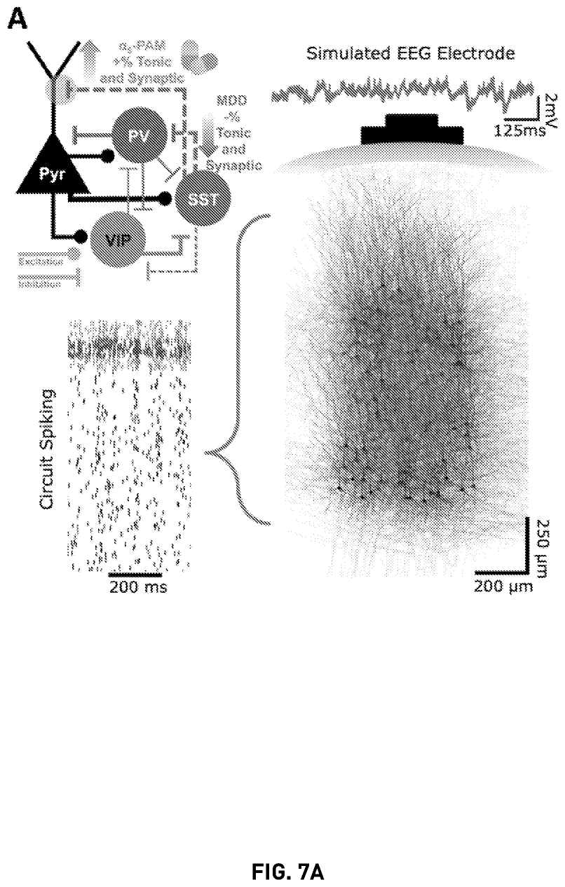

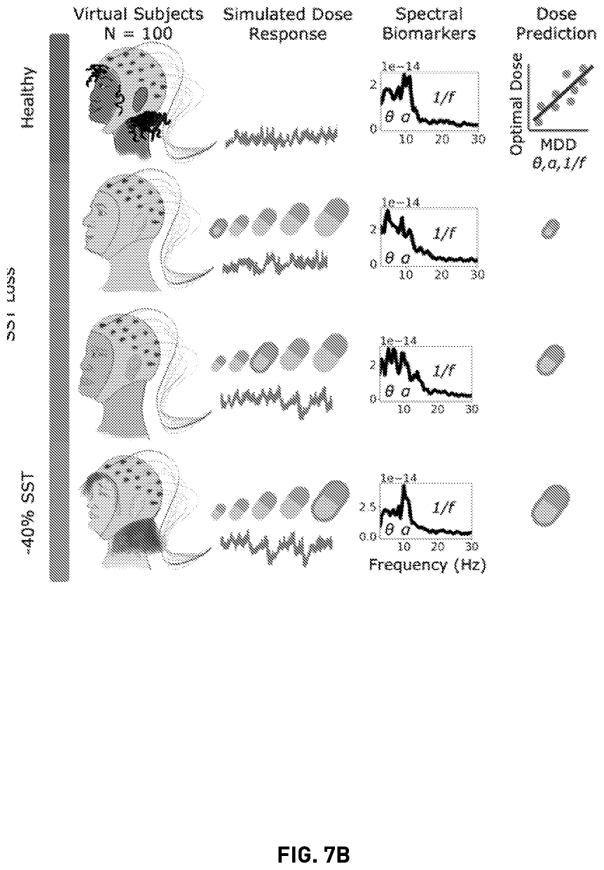

FIG. 7A illustrates simulated human layer 2/3 cortical microcircuits containing detailed models of four neuron types: Pyr, SST, PV, and VIP. These models generate voltage traces and circuit spiking, as well as simulated EEG signals. The connectivity schematic (top left) highlights the mechanisms of depression (MDD; loss of SST tonic and synaptic inhibition to all cells) and α5-PAM doses (boosted SST tonic and synaptic inhibition to Pyr neurons).

FIG. 7B schematically represents simulation of five levels of SST loss severity (0%, 10%, 20%, 30%, 40%) across 20 different circuits, representing a total of 100 different virtual subjects. For each subject a dose-response of seven α5-PAM doses ranging from 0% to 150% of the reference dose was simulated. Power spectral biomarkers, extracted from the simulated EEG signals of each virtual subject, were used to extrapolate and predict the optimal doses for restoring the metrics back to healthy ranges.

### Simulated EEG Biomarkers of Depression Severity

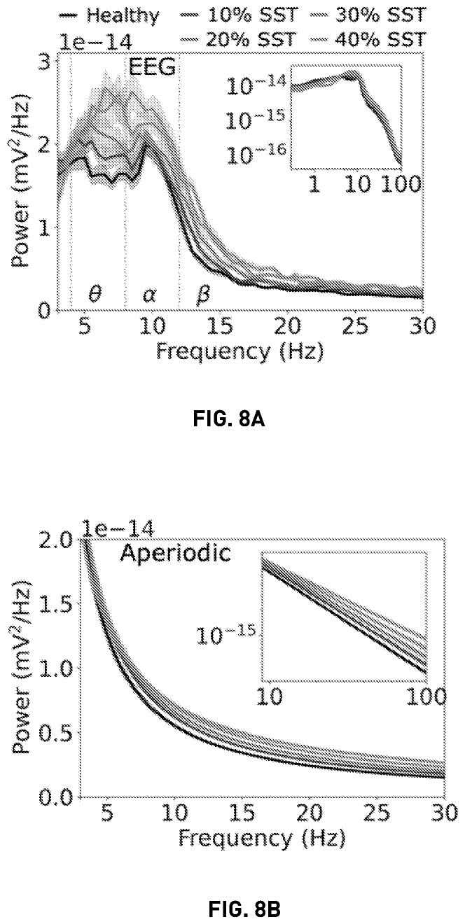

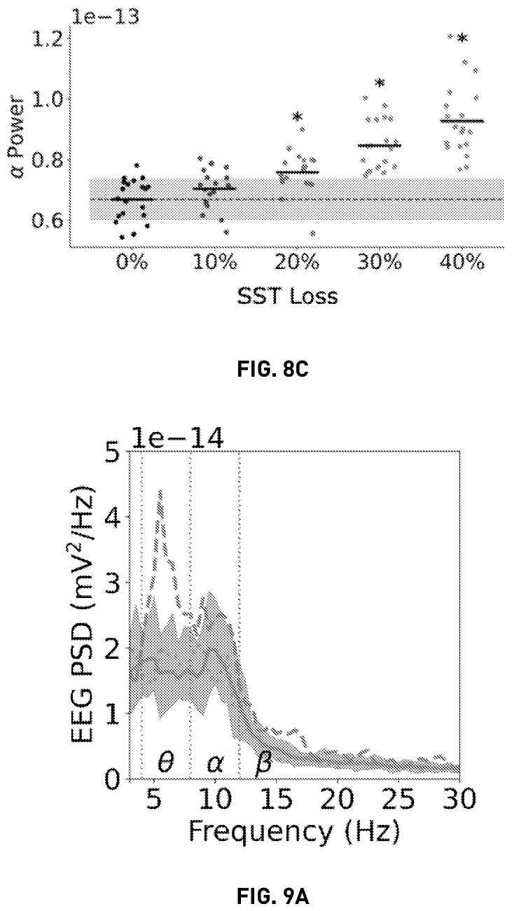

FIG. 8A is a graph of power spectral density (PSD) of simulated EEG from each severity level of SST loss (bootstrapped mean and 95% confidence intervals across circuits). Inset-PSD plotted in log scale.

FIG. 8B shows fitted aperiodic components (1/f) of the PSD for each condition (bootstrapped mean and 95% confidence intervals across circuits). Inset—1/f component plotted in log scale.

FIG. 8C shows PSD power in the alpha range (8-12 Hz) for each level of SST loss (grey—healthy standard deviation). All asterisks denote significant paired t-tests (p<0.05) with effect sizes greater than 1 when compared to healthy.

### EEG Biomarkers of Depression Severity Predict Optimal Drug Dose for Circuit Function Recovery

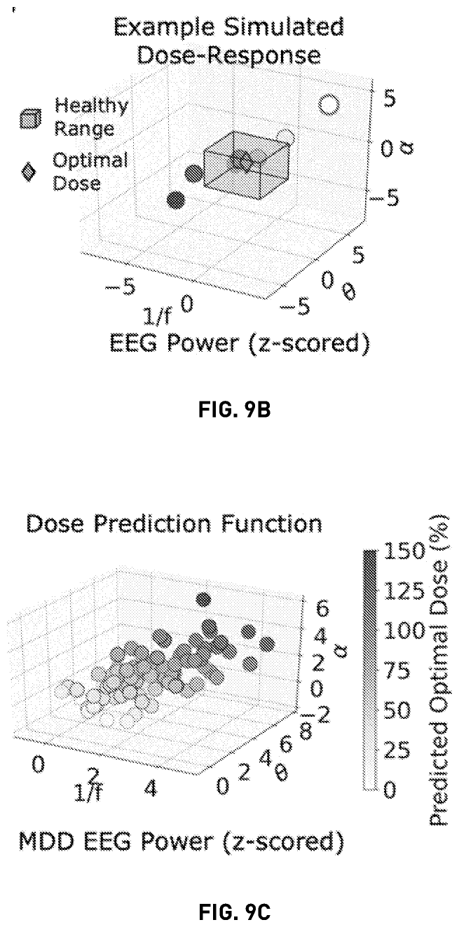

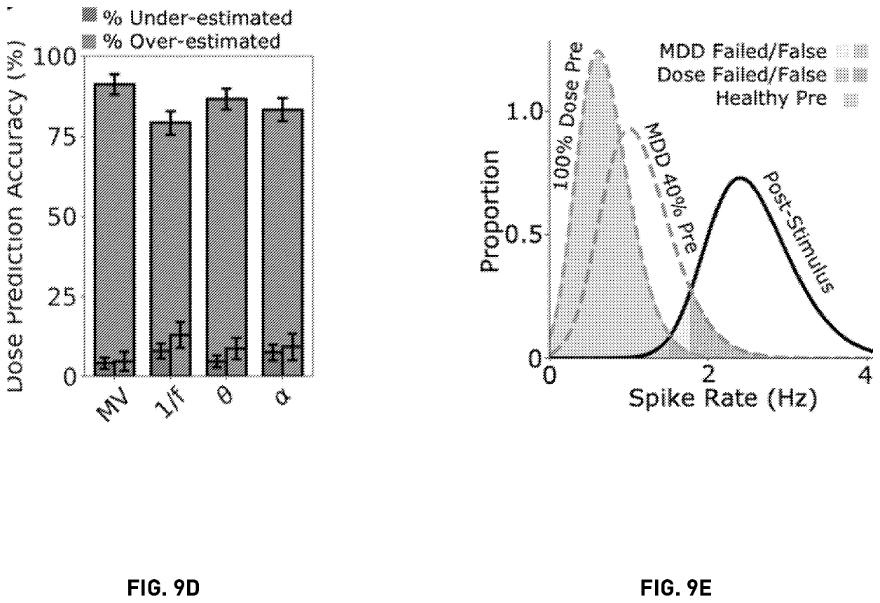

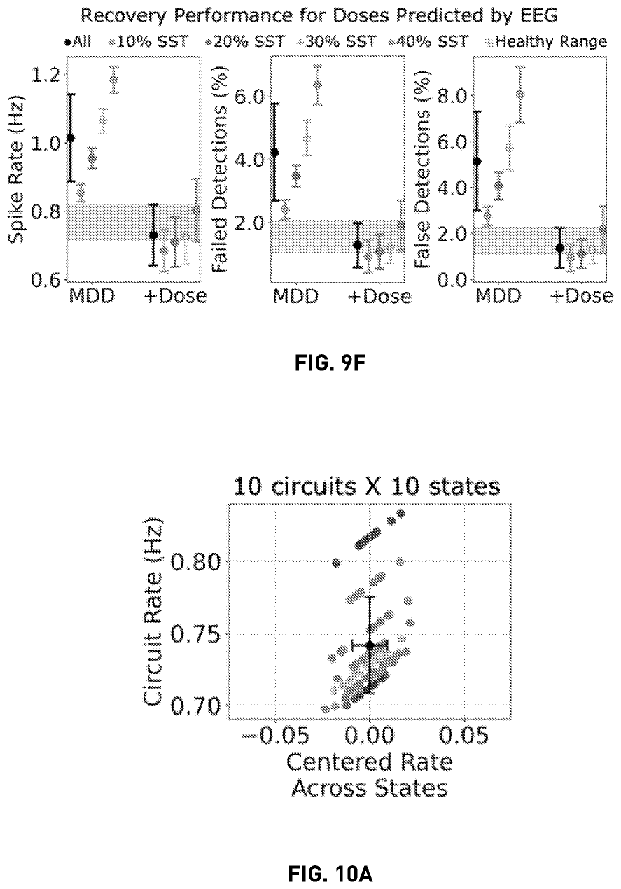

FIG. 9A shows example power spectral density (PSD) profiles for an example circuits with 30% reduced SST before and after application of the drug (dashed and dotted lines, respectively), as well as the healthy average PSD (solid line).

FIG. 9B shows dose-response for an example circuit with 30% SST interneuron inhibition loss (open circle upper right quadrant), plotted across three candidate EEG biomarkers of depression severity (1/f AUC, θ AUC, α AUC). A linear fit of the response was used to predict the optimal dose (diamond) and range with respect to the healthy mean and ranges (grey cube), respectively.

FIG. 9C shows predicted optimal doses for each circuit plotted against its EEG biomarkers in MDD before drug application.

FIG. 9D shows percent of correct dose prediction for test circuit sets using multivariate (MV) or single EEG biomarkers (1000 permutations; left inset bars=under-estimated errors; right inset bars=over-estimated errors).

FIG. 9E shows distributions of pre-stimulus firing rates from an example circuit with 40% SST loss (n=23,950 windows of 50 ms, dashed line) before (MDD 40% Pre) and after (100% Dose Pre) predicted dose, and average healthy post-stimulus firing rates (n=200 windows of 50 ms, solid black line). The overlaps between pre- and post-stimulus curves indicate failed and false signal detection errors.

FIG. 9F illustrates mean and standard deviation of spike rate (left), failed detection rates (middle), and false detection rates (right) in simulated MDD circuits before and after applying the predicted drug dose. Grey area shows the healthy range. MV dose prediction function: Optimal Dose=0.04·1/f+0.08·θ+0.13·α+0.11, where inputs metrics are z-scored PSD power relative to healthy and optimal dose is expressed as fraction relative to the reference dose. Although not available in the clinic, spike rate is provided as a ground-truth validation of the drug effects on the neuronal microcircuit.

### Drug Dose Prediction and Recovery Using Ground-Truth Circuit Spike Rates

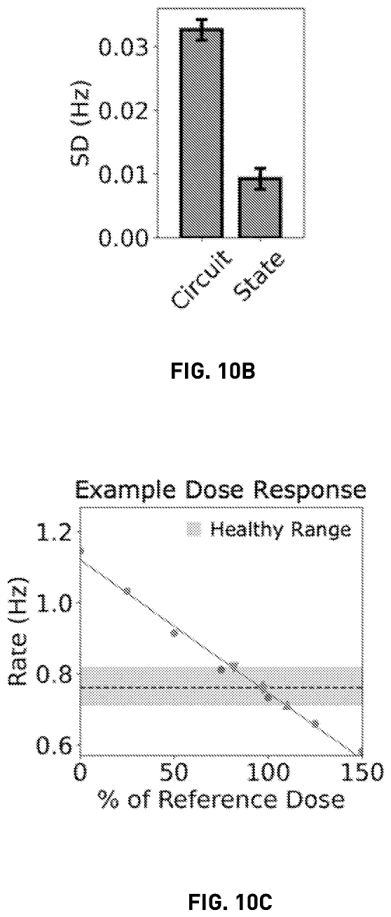

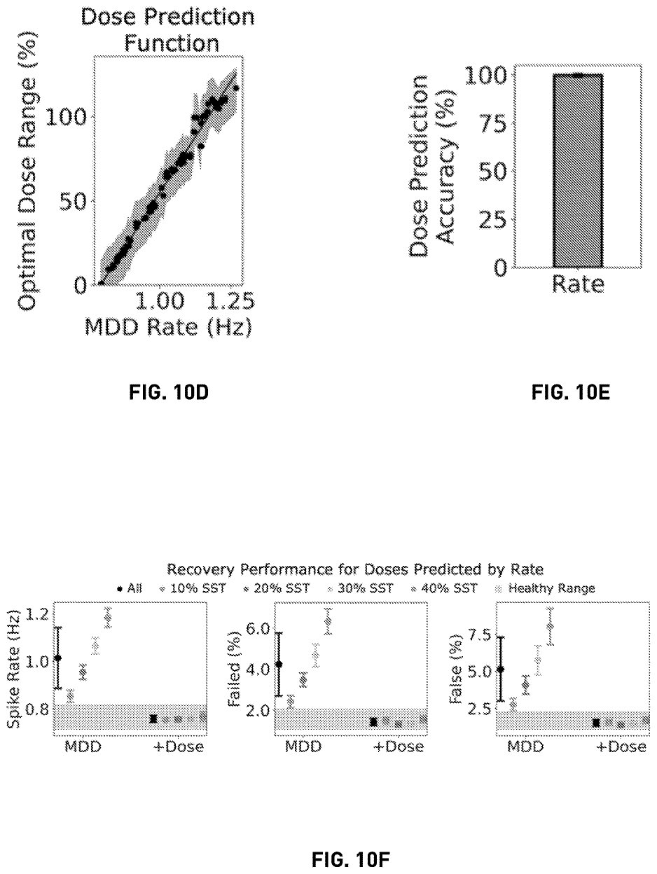

FIG. 10A shows variance due to circuit connectivity randomizations was larger than the variance due to activity state randomizations (10 healthy circuit seeds across 10 activity state seeds). Error bars show SD. Different circuits are denoted by different shading.

FIG. 10B shows average SD of spike rate across circuits or states.

FIG. 10C shows dose response for an example circuit with severity 40% SST loss, which was used to calculate the optimal dose (diamond) and optimal dose range (triangles) with respect to the healthy mean and ranges (shaded grey area).

FIG. 10D shows optimal dose (dots) and ranges (shaded area) for each circuit plotted as a function of its spike rate in MDD, before drug application. Line shows linear fit used as prediction function.

FIG. 10E shows percent of correct prediction of dose for test circuits sets (1000 permutations).

FIG. 10F shows mean and SD of spike rates (left), failed detection rates (middle), and false detection rates (right) in simulated MDD circuits and after applying predicted doses. Grey area shows the healthy range.

Simulated EEG Biomarkers Strongly Correlate with Circuit Spike Rates

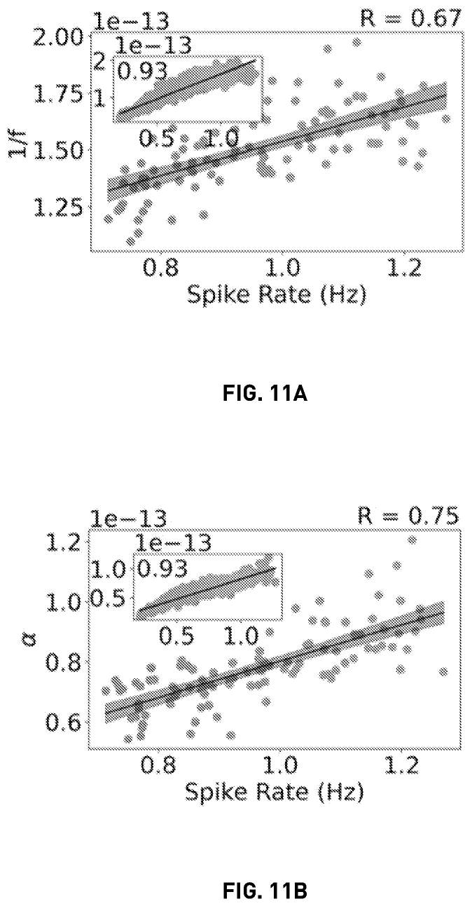

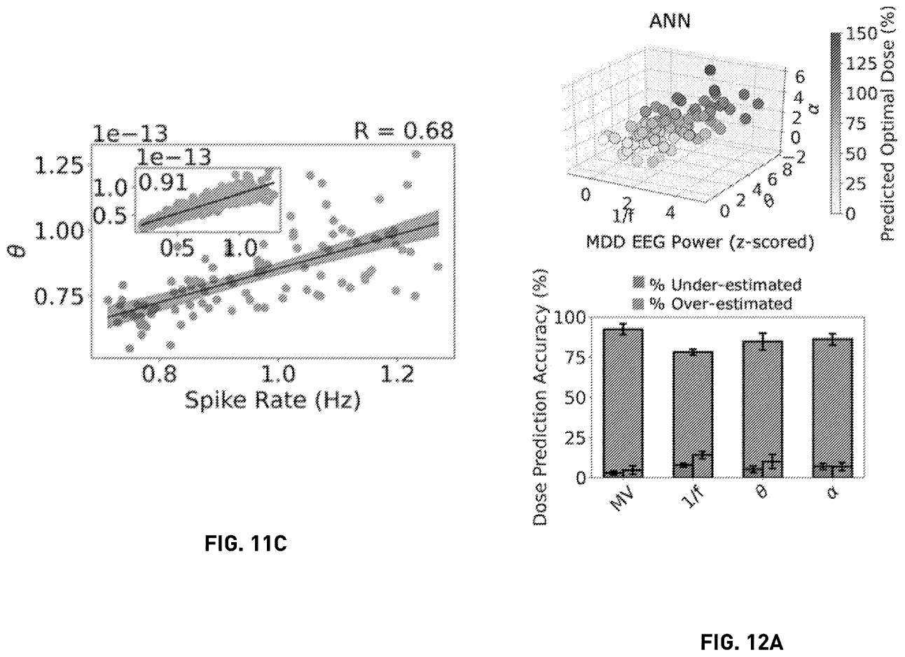

PSD metrics that showed the highest correlations with Pyr neuron spike rate across all virtual subjects are shown in FIG. 11A (1/f power), FIG. 11B (α power), and FIG. 11C (θ power) (grey dots=healthy; darker purple dots=virtual subjects with larger SST loss). Insets—same correlation but with dose application conditions included (darker blue dots=conditions with higher doses; R value shown in top left corner).

### ANN and SVM Regression Methods Generate Comparable Accuracy Scores to Linear Regression.

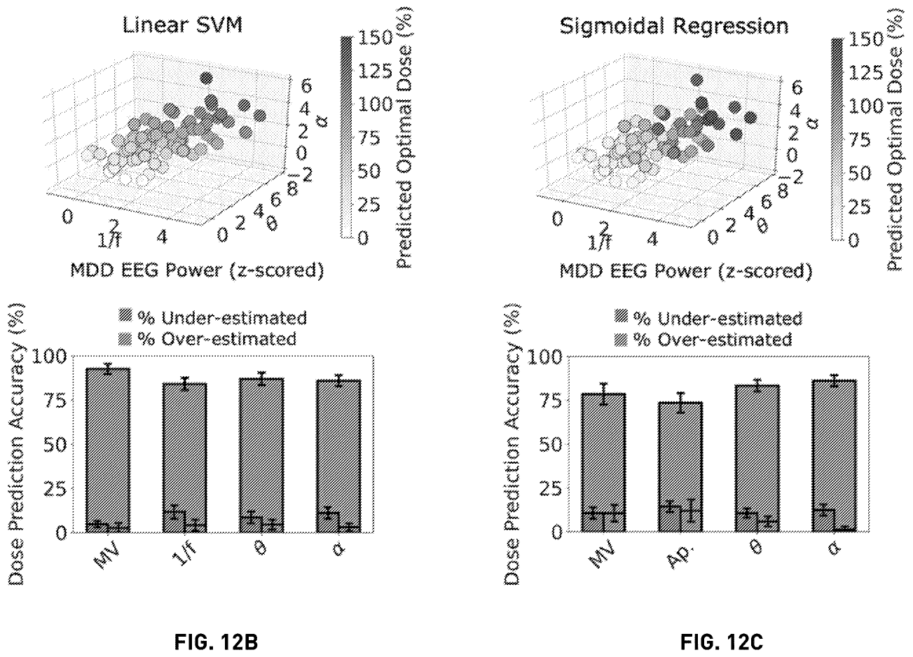

Prediction of optimal dose (top) and resulting accuracy scores (bottom) is shown using either (i) an artificial neural network (ANN) with either 3 or 1 input nodes, 9 hidden layer nodes, and 1 output node using TensorFlow (FIG. 12A); (ii) an SVM with a linear kernel, using scikit-learn module in python (FIG. 12B); or (iii) a sigmoidal regression (FIG. 12C). These results show that ANN and SVM regression methods generate comparable accuracy scores compared with linear regression.

The experiments described herein show, as a proof of concept, simulated effects of α5-PAM in human cortical microcircuit models of depression and found that this was sufficient in restoring circuit dynamics back to healthy levels. This finding alone is considerable, given that the models were constrained to simulate the effects of α5-PAM using therapeutic doses of GL-II-73, and obtained levels of recovery in stimulus processing that are on the same order of magnitude as the pro-cognitive effects in chronically stressed mice when administered GL-II-73 (T. D. Prevot et al., 2019 Novel Benzodiazepine-Like Ligands with Various Anxiolytic, Antidepressant, or Pro-Cognitive Profiles. Molecular Neuropsychiatry, 5(2), 84-97). It is also notable that the simulated effects α5-PAM do not fully restore the parameters in the depression circuits back to that of healthy levels and they do not recover any of the lost SST-mediated inhibition onto other interneurons, so it is not intuitive that α5-PAM would have been sufficient to restore circuit dynamics. Further EEG biomarkers of interest are also identified for assessing drug efficacy, highlighting recovery in theta and beta rhythm power spectral densities, as well as recovery in the aperiodic broadband shift and exponent parameters. More generally, this work provides the first framework for testing the effects of novel pharmacology in human cortical microcircuits in silico.

In addition, the results presented here establish a dose prediction function that prescribes the dose, in terms of a percent of the reference experimental dose, given EEG metrics that reflect severity of SST interneuron inhibition loss. The prediction function was based on detailed simulations of virtual subjects with different depression severity, and tested in terms of recovering EEG power, relevant brain function of signal detection from noise, relative to the ranges obtained from virtual healthy subjects. The recovery was also tested using the simulated ground-truth firing rates of neurons, which is an important property of the brain network. These results provide tools that can readily be tested in preclinical and clinical subjects, to shorten the process of dose prediction and eliminating unnecessary exploratory dose applications.

It has previously been established that enhanced theta, alpha, and lower beta frequency band powers are linked to depression diagnosis & severity, and can be used as indicators of treatment response. The present study demonstrates that α5-PAM exposure could directly recover resting state aperiodic and periodic power spectral biomarkers associated with a loss of SST-mediated inhibition in depression. By comparison, the use of non-selective PAM generated less intuitive effects on power spectral biomarkers and did not recover the power spectral profile.

This work also offers insights into why non-selective PAM do not recover circuit spiking dynamics in depression despite dampening spike rates, which was expected based previous experimental work showing the effects of benzodiazepine application in rodent cortical cultures. In fact, the small change from the MDD circuits may be a result the non-selective PAM boosting inhibition to inhibitory interneurons. In other words, the non-selective PAM both boosts inhibition to Pyr neurons while also simultaneously disinhibiting Pyr neurons. The large aperiodic upward shift in the EEG power spectral density caused by non-selective PAM may also contribute to the enhanced delta (1-5.5 Hz) that is seen in EEG signals during exposure to non-selective PAM in rodents, and is also present in slow wave sleep. These findings further highlight a lack of effectiveness of non-selective PAM at treating depression, in line with what has been suggested by previous clinical studies.

### Experimental Methods

Electrophysiology Data. We used whole-cell voltage-clamp recordings of baseline tonic inhibition current and tonic inhibition current during exposure to α5-PAM in human cortical L2/3 Pyr neurons (10 cells: 9 cells from 3 male subjects, 1 cell from 1 female subject) from patients undergoing a standard anterior temporal lobectomy or tumor resection from the frontal or temporal lobe. Written informed consent was obtained from all participants, in accordance with the Declaration of Helsinki and the University Health Network Research Ethics board.

The data was collected using surgery resection, solutions, tissue preparation, and recording equipment described in Chameh et al., 2021 Nature Communications, 12(1), 2497 and Yao et al., 2022 Cell Reports, 38(2). Neocortical tissue resected during tumor resection and anterior temporal lobectomy was immediately submerged in ice-cold (~4° C.) cutting solution and transferred to a recording chamber within 20 minutes. After sectioning the tissue, the slices were incubated for 30 min at 34° C. in standard artificial cerebrospinal fluid (aCSF) (in mM): NaCl 123, KCl 4, CaCl₂·2H₂O 1.5, MgSO₄·7H₂O 1.3, NaHCO₃ 26, NaH₂PO₄·H₂O 1.2, and D-glucose 10, pH 7.40 and bubbled with carbogen gas (95% O₂-5% CO₂) and had an osmolarity of 300-305 mOsm.

For recordings, slices were transferred to a recording chamber mounted on a fixed-stage upright microscope (Axioskop 2 FS MOT; Carl Zeiss, Germany). Slices were continually perfused at 4 ml/min with standard aCSF at 32-34° C. Whole-cell patch-clamp recordings were obtained using a Multiclamp 700 A amplifier, Axopatch 200B amplifier, and pClamp 9.2 and pClamp 10.6 data acquisition software (Axon instruments, Molecular Devices, USA). Subsequently, electrical signals were digitized at 20 kHz using a 1320X digitizer. For voltage-clamp recordings of tonic current, low-resistance patch pipettes (2-4 MΩ) were filled with a CsCl-based solution containing (in mM) 140 CsCl, 10 EGTA, 10 Hepes, 2 MgCl₂, 2 Na₂ATP, 0.3 GTP, and 5 QX314 adjusted to pH 7.3 with CsOH. The junction potential was calculated to be 4.3 mV and the holding potential was-74.3 mV after junction potential correction. In this configuration, 5 μM GABA, 25 μM AP5, 10 μM CNQX, and 10 μM CGP-35348 were first applied to generate larger GABA-dependent currents and assess baseline tonic inhibition currents while also blocking AMPA, NMDA, and GABA_{B} mediated currents. 3 μM of G-II-73 (T. D. Prevot et al., 2019) was then applied to assess tonic inhibition current in the presence of α5-PAM, followed by 50 μM of picrotoxin to block GABA_{A} mediated currents and assess endogenous current output during voltage-clamp recordings without any synaptic activity. We note that 3 μM of G-II-73 is in the therapeutic range for selectively targeting α5 subunit receptors and higher levels more strongly target α1, α2, & α3 subunit receptors (T. D. Prevot et al., 2019). The mean amplitude of baseline tonic current relative to the endogenous current generated in the picrotoxin condition, was 65.86±10.28 pA (standard error of the mean; n=10 cells). The mean amplitude of tonic inhibitory current generated during application of α5-PAM relative to the endogenous current generated in the picrotoxin condition, was 95.28±14.52 pA (standard error of the mean; n=10 cells).

GABA_{A} Receptor Positive Allosteric Modulator Models. We used a model for outwardly rectifying tonic inhibition (Bryson et al., 2020 Proceedings of the National Academy of Sciences, 117(6), 3192-3202) as well as the baseline tonic inhibition conductance values that had previously been fitted to capture the baseline tonic inhibition current magnitudes recorded in human L2/3 Pyr neurons, see electrophysiology methods above and Yao et al., 2022. The experimental conditions had been simulated by setting the inhibitory chloride reversal potential to −5 mV (i.e. consistent with the experimental solutions), setting the holding potential to −75 mV in voltage-clamp mode, and tuning the tonic inhibition conductance on all Pyr neuron somatic and dendritic compartments (G_{tonic}: 0.938 mS/cm²) to reproduce the target experimental tonic inhibition current amplitude. The same G_{tonic} value was used for the interneurons since the total tonic inhibition current recorded in interneurons is similar to that of Pyr neurons after correcting for cell capacitance. We used the same simulation method to estimate the G_{tonic} change on Pyr neuron apical dendrites during application of α5-PAM (G_{tonic}: 1.498 mS/cm²), again using the experimental tonic inhibition currents as target magnitudes. All simulated current magnitudes were calculated relative to the endogenous current magnitude generated in the condition where G_{tonic} is set to 0 mS/cm², consistent with the picrotoxin condition in the experimental methodology. In our depression circuit simulations, the changes in G_{tonic} and SST→Pyr synaptic conductance (G_{SST→Pyr}) due to α5-PAM exhibit the same G_{tonic} percent change (+60%) that we measured in the in vitro healthy Pyr neuron simulations. For different α5-PAM doses, we simulated different fractional levels of this 60% conductance changes, ranging from 25% to 150%, where 100% is the reference dose that we initially fitted. In separate simulations, we also modeled the action of non-selective GABA_{A} receptor positive allosteric modulators, where we instead applied the 60% percent change in conductance to all tonic inhibition and synaptic inhibition parameters in the microcircuit.

Failed/False Signal Detection Rates. We computed error rates in stimulus processing with our microcircuit models as in Yao et al., 2022, where we first fit the pre-stimulus firing rate distributions (computed using a 50 ms sliding window, sliding in 1 ms intervals, over a 2 s pre-stimulus period) to skewed normal distributions for each of the 200 randomized microcircuits. We then computed the fitted post-stimulus firing rate distribution (computed in the 5-55 ms period post-stimulus) across all the 200 randomized microcircuits. The intersection points between each pre-stimulus distribution and the post-stimulus distribution were identified as the stimulus detection threshold points. Probability of false detections was computed as the integral of the pre-stimulus distribution above the detection threshold divided by the integral of the entire pre-stimulus distribution. Likewise, the probability of failed detections was computed as the integral of the post-stimulus distribution under the detection threshold divided by the integral of the entire post-stimulus distribution.

Simulated Microcircuit EEG and Power Spectral Analyses. Dipole moments and corresponding EEG time series data generated by the microcircuit models was simulated as described in Hagen et al., 2018 Frontiers in Neuroinformatics, 12; and Mazza et al., 2023 PLOS Computational Biology 19 (4), e1010986. Specifically, a four-sphere volume conductor model representing grey matter, cerebrospinal fluid, skull, and scalp was used, each with radii of 79 mm, 80 mm, 85 mm, and 90 mm, respectively. The model assumes homogeneous, isotropic, and linear (frequency-independent) conductivity. The conductivity for each sphere was 0.047 S/m, 1.71 S/m, 0.02 S/m, and 0.41 S/m, respectively. EEG power spectral density was computed using Welch's method from the SciPy python module. Power spectral features were quantified by computing the integral of the power spectral densities for theta (4-8 Hz), alpha (8-12 Hz), and lower beta (12-21 Hz) range frequencies.

The EEG power spectral densities (in the 2-30 Hz range) were decomposed into aperiodic and periodic components using an algorithmic parameterization method as described in Donoghue et al., 2020 Nature Neuroscience, 23(12), 1655-1665. Using this approach, the aperiodic component was defined as a 1/f function parameterized by vertical offset and exponent parameters. As an additional quantification metric, the integral of the broadband (3-30 Hz) frequency range in the aperiodic component (or area-under the curve, AUC) was computed. Overlying the aperiodic component, the periodic oscillatory component was defined by fits with up to 4 Gaussian peaks, which were defined by center frequency, bandwidth (min: 2 Hz, max: 12 Hz), and power magnitude (relative peak threshold: 2, minimum peak height: 0.5). Periodic features were quantified by computing the integral of the periodic component for theta (4-8 Hz), alpha (8-12 Hz), and lower beta (12-21 Hz) range frequencies.

We also computed the power spectral density of Pyr neuron population spiking by converting the spike times into binary spike train vectors, which were then summed across all Pyr neurons. Power spectral density was then computed from the summed spike train vectors using Welch's method, see Guet-McCreight & Skinner, 2019 PLOS ONE, 14(1), e0209429; Yao et al., 2022 Cell Reports, 38(2). For both Pyr neuron spiking and EEG, we computed power spectral density with nperseg=140,000 sampling points, which is equivalent to 3.5 s time windows. For spiking and EEG power spectral density vectors, as well as the aperiodic and periodic vectors, across random seeds we computed the bootstrapped (100 iterations) means and 95% confidence intervals for each frequency.

While the invention has been described by means of specific embodiments and applications thereof, modifications and variations could be made thereto by those skilled in the art without departing from the scope set forth in the claims.

The present invention is set forth in various levels of detail. In certain instances, details not necessary for one of ordinary skill in the art to understand the invention may have been omitted.

Section headings are for organizational purposes only and are not to be construed as limiting the subject matter described.

The terminology used herein is for the purpose of describing particular embodiments only and is not intended to be limiting beyond the scope of the appended claims. An “embodiment” may refer to an illustrative representation of a method or article in which a disclosed concept or feature may be provided or embodied, or a representation of a manner in which a concept or feature may be provided or embodied. Such illustrated embodiments are to be understood as examples (unless otherwise stated), and other manners of embodying the described concepts or features, such as may be understood by one of ordinary skill in the art upon learning the concepts or features from the present disclosure, are within the scope of the disclosure. Accordingly, disclosed embodiments are to be considered in all respects as illustrative and not restrictive, the scope of the claimed subject matter being indicated by the appended claims, and not limited to the foregoing description or particular embodiments or arrangements described or illustrated herein. It is intended that the present subject matter covers such modifications and variations as come within the scope of the appended claims and their equivalents.

Unless defined otherwise, technical terms used herein are to be understood as commonly understood by one of ordinary skill in the art to which the disclosure belongs.

The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein.

The term “about” when used before a numerical designation, e.g., temperature, time, amount, concentration, and such other, including a range, indicates approximations which may vary by (+) or (−) 10%, 5%, 1%, or any subrange or subvalue there between. Preferably, the term “about” means that the value may vary by +/−10%.

The term “comprises/comprising” does not exclude the presence of other elements, components, features, regions, integers, steps, operations, etc. Additionally, although individual features may be included in different claims, these may possibly advantageously be combined, and the inclusion in different claims does not imply that a combination of features is not feasible and/or advantageous. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

The term “subject” refers to a mammal, for example a mouse, a rat, a dog, a guinea pig, a non-human primate, or a human. In some embodiments, the subject is a human. The term “patient” refers to a human subject.

The terms “treatment,” “treating,” “treat,” and the like, refer to obtaining a desired pharmacologic and/or physiologic effect from a therapy in relation to a disease or disorder. The effect is therapeutic in terms of achieving a clinical response, which may be partial or complete, and may alleviate or reduce the severity of one or more symptoms attributable to the disease or disorder being treated. “Treating” can also include inhibiting or slowing the progression of a disease or disorder.

1. A method of determining a therapeutic dose of a α5-GABA_{A} receptor positive allosteric modulator (α5-PAM) for a subject in need thereof, the method comprising
obtaining or receiving an electroencephalography (EEG) signal from the subject;
performing, on a processor, a power spectral density (PSD) calculation on the signal to obtain a raw PSD;
decomposing, on a processor, the raw PSD into its aperiodic and periodic components;
determining, based on the raw PSD, the aperiodic components, the periodic components, and a dose prediction function, the therapeutic dose of an α5-PAM for the subject.

2. A method of treating a subject in need thereof with a α5-GABA_{A} receptor positive allosteric modulator (α5-PAM), the method comprising
obtaining or receiving an electroencephalography (EEG) signal from the subject in a pre-treatment condition and at least one subsequent post-treatment condition, wherein the pre-treatment condition corresponds to pre-treatment with an α5-PAM and the post-treatment condition corresponds to after administration of at least one dose of the α5-PAM to the subject;
performing, on a processor, a power spectral density (PSD) calculation on the signal obtained or received for each condition to obtain raw PSD for each condition;
decomposing, on a processor, the raw PSD into its aperiodic and periodic components for each condition;
determining, based on the raw PSD, the aperiodic components, the periodic components, and a dose prediction function, a predicted dose for the subject for each condition;
comparing the predicted dose at each condition, wherein a decrease in predicted dose indicates that the α5-PAM is having a therapeutic effect; and
administering a second or further dose of the α5-PAM to the subject and repeating until the predicted dose is zero, or smaller than a predetermined amount.

3. The method of claim 2 wherein the aperiodic components comprise broadband AUC and exponent (χ).

4. The method of claim 2 wherein the raw PSD and periodic components comprise the power measured by integral of one or more PSD frequency bands selected from theta, alpha, and beta frequency bands, and/or sub-bands thereof.

5. The method of claim 2 wherein the dose prediction function is derived in silico from virtual subjects of different depression severity.

6. The method of claim 5, wherein the dose prediction function is applied to a pre-treatment condition and then a post-treatment condition of the subject, optionally wherein the dose prediction function is applied iteratively across different treatments.

7. The method of claim 2 wherein the EEG signal is obtained over a period of time.

8. The method of claim 7, wherein the period of time is from 2-5 minutes, from 5-15 minutes, from 15-30 minutes, from 30-60 minutes, or from 60-120 minutes.

9. The method of claim 7, wherein the period of time is about 5, 10, 15, 20, 25, 30, 35, 40, 45, 50, 60, 90, or 120 minutes.

10. The method of claim 2 wherein the α5-PAM is GL-II-73 or a derivative thereof.

11. The method of claim 2 wherein the subject in need is one diagnosed with a neuropsychiatric disorder, a neurological disorder, a neurodegenerative disorder, or any combination thereof.

12. The method of claim 11, wherein the neuropsychiatric disorder is bipolar disorder or schizophrenia.

13. The method of claim 11, wherein the neurological disorder is epilepsy or other brain related disorders.

14. The method of claim 11, wherein the neurodegenerative disorder is Alzheimer's disease or dementia.

15. The method of claim 2 wherein the subject in need is one diagnosed with depression.

16. The method of claim 2 wherein the subject in need is one diagnosed with major depressive disorder (MDD).

17. The method of claim 15 the depression is treatment resistant.
